SEQUENCE LISTING (38 sequences)

SEQ ID NO: 1 - DNA, 1122 bp, Homo sapiens
atgtccccgtgtcctccccagcagagcaggaacagggtgatacagctgtccacttcagagctaggagagatggaactgacttggcaggagatcatgtccatcaccgagctgcagggtctgaatgctccaagtgagccatcatttgagccccaagccccagctccataccttggacctccaccacccacaacttactgcccctgctcaatccacccagattctggcttcccacttcctccaccaccttatgagctcccagcatccacatcccatgtcccagatcccccatactcctatggcaacatggccataccagtctccaagccactgagcctctcaggcctgctcagtgagccgctccaagaccccttagccctcctggacattgggctgccagcagggccacctaagccccaagaagacccagaatccgactcaggattatccctcaactatagcgatgctgaatctcttgagctggaggggacagaggctggtcggcggcgcagcgaatatgtagagatgtacccagtggagtacccctactcactcatgcccaactccttggcccactccaactataccttgccagctgctgagacccccttggccttagagccctcctcaggccctgtgcgggctaagcccactgcacggggggaggcagggagtcgggatgaacgtcgggccttggccatgaagattccttttcctacggacaagattgtcaacttgccggtagatgactttaatgagctattggcaaggtacccgctgacagagagccagctagcgctagtccgggacatccgacgacggggcaaaaacaaggtggcagcccagaactgccgcaagaggaagctggaaaccattgtgcagctggagcgggagctggagcggctgaccaatgaacgggagcggcttctcagggcccgcggggaggcagaccggaccctggaggtcatgcgccaacagctgacagagctgtaccgtgacattttccagcaccttcgggatgaatcaggcaacagctactctcctgaagagtacgcgctgcaacaggctgccgatgggaccatcttccttgtgccccgggggaccaagatggaggccacagactga

SEQ ID NO: 2 - DNA, 477 bp, Homo sapiens
atgagccacgggaagggaaccgacatgctcccggagatcgccgccgccgtgggcttcctctccagcctcctgaggacccggggctgcgtgagcgagcagaggcttaaggtcttcagcggggcgctccaggaggcactcacagagcactacaaacaccactggtttcccgaaaagccgtccaagggctccggctaccgctgcattcgcatcaaccacaagatggaccccatcatcagcagggtggccagccagatcggactcagccagccccagctgcaccagctgctgcccagcgagctgaccctgtgggtggacccctatgaggtgtcctaccgcattggggaggacggctccatctgcgtcttgtacgaggaggccccactggccgcctcctgtgggctcctcacctgcaagaaccaagtgctgctgggccggagcagcccctccaagaactacgtgatggcagtctccagctag

SEQ ID NO: 3 - DNA, 789 bp, Homo sapiens
atgctcgccctggaggctgcacagctcgacgggccacacttcagctgtctgtacccagatggcgtcttctatgacctggacagctgcaagcattccagctaccctgattcagagggggctcctgactccctgtgggactggactgtggccccacctgtcccagccaccccctatgaagccttcgacccggcagcagccgcttttagccacccccaggctgcccagctctgctacgaaccccccacctacagccctgcagggaacctcgaactggcccccagcctggaggccccggggcctggcctccccgcataccccacggagaacttcgctagccagaccctggttcccccggcatatgccccgtaccccagccctgtgctatcagaggaggaagacttaccgttggacagccctgccctggaggtctcggacagcgagtcggatgaggccctcgtggctggccccgaggggaagggatccgaggcagggactcgcaagaagctgcgcctgtaccagttcctgctggggctactgacgcgcggggacatgcgtgagtgcgtgtggtgggtggagccaggcgccggcgtcttccagttctcctccaagcacaaggaactcctggcgcgccgctggggccagcagaaggggaaccgcaagcgcatgacctaccagaagctggcgcgcgccctccgaaactacgccaagaccggcgagatccgcaaggtcaagcgcaagctcacctaccagttcgacagcgcgctgctgcctgcagtccgccgggcctga

SEQ ID NO: 4 - DNA, 1281 bp, Homo sapiens
atgtgtgaccggaatggtggtcggcggcttcgacagtggctgatcgagcagattgacagtagcatgtatccaggactgatttgggagaatgaggagaagagcatgttccggatcccttggaaacacgctggcaagcaagattataatcaggaagtggatgcctccatttttaaggcctgggcagtttttaaagggaagtttaaagaaggggacaaagctgaaccagccacttggaagacgaggttacgctgtgctttgaataagagcccagattttgaggaagtgacggaccggtcccaactggacatttccgagccatacaaagtttaccgaattgttcctgaggaagagcaaaaatgcaaactaggcgtggcaactgctggctgcgtgaatgaagttacagagatggagtgcggtcgctctgaaatcgacgagctgatcaaggagccttctgtggacgattacatggggatgatcaaaaggagcccttccccgccggaggcctgtcggagtcagctccttccagactggtgggcgcagcagcccagcacaggcgtgccgctggtgacggggtacaccacctacgacgcgcaccattcagcattctcccagatggtgatcagcttctactatgggggcaagctggtgggccaggccaccaccacctgccccgagggctgccgcctgtccctgagccagcctgggctgcccggcaccaagctgtatgggcccgagggcctggagctggtgcgcttcccgccggccgacgccatccccagcgagcgacagaggcaggtgacgcggaagctgttcgggcacctggagcgcggggtgctgctgcacagcagccggcagggcgtgttcgtcaagcggctgtgccagggccgcgtgttctgcagcggcaacgccgtggtgtgcaaaggcaggcccaacaagctggagcgtgatgaggtggtccaggtcttcgacaccagccagttcttccgagagctgcagcagttctataacagccagggccggcttcctgacggcagggtggtgctgtgctttggggaagagtttccggatatggcccccttgcgctccaaactcattctcgtgcagattgagcagctgtatgtccggcaactggcagaagaggctgggaagagctgtggagccggctctgtgatgcaggcccccgaggagccgccgccagaccaggtcttccggatgtttccagatatttgtgcctcacaccagagatcatttttcagagaaaaccaacagatcaccgtctaa

SEQ ID NO: 5 - DNA, 867 bp, Homo sapiens
atggagcctccggaccagtgtagccagtatatgaccagcttgctcagccctgcagtcgacgacgagaaagaactacaggatatgaatgctatggtgctgtcgcttactgaagaggtcaaagaggaggaagaggatgcacagcctgagcctgagcaaggcacagcagcaggagaaaagttaaagtcggcaggagcccaaggcggagaagaaaaagatggcggcggagaagaaaaagatggcggcggcgccggagttcctggccacctatgggaaggagacctcgagggcaccagcggcagcgatggcaacgttgaggacagcgaccagagcgagaaggaacctgggcagcagtattcgcgcccacagggcgccgtcggggggctggagcctggcaacgcgcagcagcccaacgtccacgccttcaccccattgcagctgcaggagctggagcgcattttccaacgcgagcagttccccagtgagttcctgcgaaggaggctggcaagaagcatgaatgtgactgaactcgcagtgcagatttggtttgagaatagaagagccaaatggaggagacatcagagggcattaatggcaagaaacatgctgcccttcatggcagtgggccagcctgtcatggtaaccgcagctgaggccataacggcacccttgttcatcagcgggatgagagatgattacttctgggaccacagccattccagcagcctgtgtttccccatgccaccctttcctcctccgtccttgccccttccactcatgcttcttccacctatgccacccgctggccaggctgaatttggcccattcccttttgttatcgtgccttctttcacattccccaatgtctaa

SEQ ID NO: 6 - DNA, 1530 bp, Homo sapiens
atggaagatatacaaacaaatgcggaactgaaaagcactcaggagcagtctgtgcccgcagaaagtgcagcggttttgaatgactacagtttaaccaaatctcatgaaatggaaaatgtggacagtggagaaggcccagccaatgaagatgaagacataggagatgattcaatgaaagtgaaagatgaatacagtgaaagagatgagaatgttttaaagtcagaacccatgggaaatgcagaagagcctgaaatcccttacagctattcaagagaatataatgaatatgaaaacattaagttggagagacatgttgtctcattcgatagtagcaggccaaccagtggaaagatgaactgcgatgtgtgtggattatcctgcatcagcttcaatgtcttaatggttcataagcgaagccatactggtgaacgcccattccagtgtaatcagtgtggggcatcttttactcagaaaggtaacctcctccgccacattaaactgcacacaggggaaaaaccttttaagtgtcacctctgcaactatgcatgccaaagaagagatgcgctcacggggcatcttaggacacattctgtggagaaaccctacaaatgtgagttttgtggaaggagttacaagcagagaagttcccttgaggagcacaaggagcgctgccgtacatttcttcagagcactgacccaggggacactgcaagtgcggaggcaagacacatcaaagcagagatgggaagtgaaagagctctcgtactggacagattagcaagcaatgtggcaaaacgaaaaagctcaatgcctcagaaattcattggtgagaagcgccactgctttgatgtcaactataattcaagttacatgtatgagaaagagagtgagctcatacagacccgcatgatggaccaagccatcaataacgccatcagctatcttggcgccgaagccctgcgccccttggtccagacaccgcctgctcccacctcggagatggttccagttatcagcagcatgtatcccatagccctcacccgggctgagatgtcaaacggtgcccctcaagagctggaaaagaaaagcatccaccttccagagaagagcgtgccttctgagagaggcctctctcccaacaatagtggccacgactccacggacactgacagcaaccatgaagaacgccagaatcacatctatcagcaaaatcacatggtcctgtctcgggcccgcaatgggatgccacttctgaaggaggttccccgctcttacgaactcctcaagcccccgcccatctgcccaagagactccgtcaaagtgatcaacaaggaaggggaggtgatggatgtgtatcggtgtgaccactgccgcgtcctcttcctggactatgtgatgttcacgattcacatgggctgccacggcttccgtgaccctttcgagtgtaacatgtgtggatatcgaagccatgatcggtatgagttctcgtctcacatagccagaggagaacacagagccctgctgaagtga

SEQ ID NO: 7 - DNA, 1068 bp, Homo sapiens
atggcgctgagtgaacccatcctgccgtccttctccactttcgccagcccgtgccgcgagcgcggcctgcaggagcgctggccgcgcgccgaacccgagtccggcggcaccgacgacgacctcaacagcgtgctggacttcatcctgtccatggggctggatggcctgggcgccgaggccgccccggagccgccgccgccgcccccgccgcctgcgttctattaccccgaacccggcgcgcccccgccctacagcgcccccgcgggtggcctggtgtctgagctgctgcgacccgagctggatgcgccgccggggcccgcactgcacggccgctttctgctggcgccgcccggccgcctggtcaaggccgagccccctgaagcggacggcggcggcggctacggctgcgcccccgggctgacccgtggaccgcgcggcctcaagcgcgagggcgccccgggcccggcggcttcgtgcatgcgaggtcccgggggccgccccccgccgccgcccgacacaccgccgctcagccccgacggccccgcgcgcctgcccgcgcccggtccgcgcgcctccttcccgccgcctttcggtggccctggtttcggcgcgcccgggcccggcctgcattacgcgccgcctgcgcccccagccttcggtctcttcgacgacgcggccgccgccgcggcagccctgggcctggcgccccccgccgcccgcggtctcctcacgccgcctgcgtccccgctggagctgctggaggccaagccaaagcgcggccgccgctcttggccccgcaaacgcaccgccactcacacctgcagctacgcgggctgcggcaagacctacaccaagagttcgcatctgaaggcgcatctgcgcacgcacacaggtgagaagccctaccactgcaactgggacggctgcggctggaagtttgcgcgctcagacgagctcacgcgccactaccgaaagcacacgggccaccggccattccagtgccatctgtgcgatcgtgccttctcgcgctccgatcacctggcgctgcacatgaaacggcacatgtag

SEQ ID NO: 8 - DNA, 996 bp, Homo sapiens
atgaccgagcggccgccgagcgaggcggctcgcagtgacccccagctagagggacgggacgcggccgaggccagcatggcccccccgcacctggtcctgctgaacggcgtcgccaaggagacgagccgcgcggccgcagcggagcccccagtcatcgaactgggcgcgcgcggaggcccggggggcggccctgccggtgggggcggcgccgcgagagacttaaagggccgcgacgcggcgacggccgaagcgcgccatcgggtgcccaccaccgagctgtgcagacctcccgggcccgccccggcccccgcgcccgcctcggttacagcggagctgcccggcgacggccgcatggtgcagctgagtcctcccgcgctggctgcccccgccgcccccggccgcgcgctgctctacagcctcagccagccgctggcctctctcggcagcgggttctttggggagccggatgccttccctatgttcaccaccaacaatcgagtgaagaggagaccttccccctatgagatggagattactgatggtccccacaccaaagttgtgcggcgtatcttcaccaacagccgggagcgatggcggcagcagaatgtgaacggggcctttgccgagctccgcaagctgatccccacacatcccccggacaagaagctcagcaagaatgagatcctccgcctggccatgaagtatatcaacttcttggccaagctgctcaatgaccaggaggaggagggcacccagcgggccaagactggcaaggaccctgtggtgggggctggtgggggtggaggtgggggagggggcggcgcgcccccagatgacctcctgcaagacgtgctttcccccaactccagctgcggcagctccctggatggggcagccagcccggacagctacacggaggagcccgcgcccaagcacacggcccgcagcctccatcctgccatgctgcctgccgccgatggagccggccctcggtga

SEQ ID NO: 9 - DNA, 783 bp, Homo sapiens
atgaatagcgcagcgggattctcacacctagaccgtcgcgagcgggttctcaagttaggggagagtttcgagaagcagccgcgctgcgccttccacactgtgcgctatgacttcaaacctgcttctattgacacttcttctgaaggataccttgaggttggtgaaggtgaacaggtgaccataactctgccaaatatagaaggttcaactccaccagtaactgttttcaaaggttcaaaaaaaccttacttaaaagaatgcattttgattattaaccatgatactggagaatgtcggctagaaaaactcagcagcaacatcactgtaaaaaaaacaagagttgaaggaagcagtaaaattcagtatcgtaaagaacaacagcaacaacaaatgtggaattcagccaggactcccaatcttgtaaaacattctccatctgaagataagatgtccccagcatctccaatagatgatatcgaaagagaactgaaggcagaagctagtctaatggaccagatgagtagttgtgatagttcatcagattccaaaagttcatcatcttcaagtagtgaggatagttctagtgactcagaagatgaagattgcaaatcctctacttctgatacagggaattgtgtctcaggacatcctaccatgacacagtacaggattcctgatatagatgccagtcataatagatttcgagacaacagtggccttctgatgaatactttaagaaatgatttgcagctgagtgaatcaggaagtgacagtgatgactga

SEQ ID NO: 10 - DNA, 993 bp, Homo sapiens
atgccacgctccttcctggtgaagagcaagaaggctcacacctaccaccagccccgtgtgcaggaagatgaaccgctctggcctcctgcccttaccccggtgcccagagaccaggctccaagcaacagccctgtccttagcactctattcccaaaccagtgcctggactggaccaacctcaaacgagagccggagctggagcaggaccagaacttggccaggatggccccggcaccagagggccccattgtgctgtcccgaccccaggatggggactctccactgtccgactcacccccattctacaagcctagcttctcctgggacaccttggccacaacctatggccacagctaccggcaggccccctccaccatgcagtcagccttcctggagcactccgtcagcctgtacggcagtcctcttgtgcccagcactgagcccgccttggacttcagcctccgctactccccaggcatggatgcgtaccactgtgtgaagtgcaacaaggtcttctccacccctcacgggctcgaagtgcatgtgcgacgctcccatagtgggacccggcccttcgcctgtgacatctgcggcaaaaccttcggccacgctgtgagcctggagcagcacacgcacgtccactcccaggagcgcagcttcgagtgccgcatgtgcggcaaggccttcaagcgctcgtccacgctgtccacccacctgctcatccactcagacacgcggccctacccctgccagttctgcggcaagcgtttccaccagaagtccgacatgaagaagcacacctacatccacacaggtgagaagccgcacaagtgccaggtgtgcggaaaggccttcagccagagctccaacctcatcacccacagccgcaagcacacaggcttcaagcccttcagctgtgagctgtgcaccaaaggcttccagcgcaaggtggacctgcggcggcaccgcgagagccagcacaatctcaagtga

SEQ ID NO: 11 - DNA, 1242 bp, Homo sapiens
atggagttccctggcctggggtccctggggacctcagagcccctcccccagtttgtggatcctgctctggtgtcctccacaccagaatcaggggttttcttcccctctgggcctgagggcttggatgcagcagcttcctccactgccccgagcacagccaccgctgcagctgcggcactggcctactacagggacgctgaggcctacagacactccccagtctttcaggtgtacccattgctcaactgtatggaggggatcccagggggctcaccatatgccggctgggcctacggcaagacggggctctaccctgcctcaactgtgtgtcccacccgcgaggactctcctccccaggccgtggaagatctggatggaaaaggcagcaccagcttcctggagactttgaagacagagcggctgagcccagacctcctgaccctgggacctgcactgccttcatcactccctgtccccaatagtgcttatgggggccctgacttttccagtaccttcttttctcccaccgggagccccctcaattcagcagcctattcctctcccaagcttcgtggaactctccccctgcctccctgtgaggccagggagtgtgtgaactgcggagcaacagccactccactgtggcggagggacaggacaggccactacctatgcaacgcctgcggcctctatcacaagatgaatgggcagaacaggcccctcatccggcccaagaagcgcctgattgtcagtaaacgggcaggtactcagtgcaccaactgccagacgaccaccacgacactgtggcggagaaatgccagtggggatcccgtgtgcaatgcctgcggcctctactacaagctacaccaggtgaaccggccactgaccatgcggaaggatggtattcagactcgaaaccgcaaggcatctggaaaagggaaaaagaaacggggctccagtctgggaggcacaggagcagccgaaggaccagctggtggctttatggtggtggctgggggcagcggtagcgggaattgtggggaggtggcttcaggcctgacactgggccccccaggtactgcccatctctaccaaggcctgggccctgtggtgctgtcagggcctgttagccacctcatgcctttccctggacccctactgggctcacccacgggctccttccccacaggccccatgccccccaccaccagcactactgtggtggctccgctcagctcatga

SEQ ID NO: 12 - DNA, 1089 bp, Homo sapiens
atggccacagccgagaccgccttgccctccatcagcacactgaccgccctgggccccttcccggacacacaggatgacttcctcaagtggtggcgctccgaagaggcgcaggacatgggcccgggtcctcctgaccccacggagccgcccctccacgtgaagtctgaggaccagcccggggaggaagaggacgatgagaggggcgcggacgccacctgggacctggatctcctcctcaccaacttctcgggcccggagcccggtggcgcgccccagacctgcgctctggcgcccagcgaggcccccggggcgcaatatccgccgccgcccgagactctgggcgcatatgctggcggcccggggctggtggctgggcttttgggttcggaggatcactcgggttgggtgcgccctgccctgcgagcccgggctcccgacgccttcgtgggcccagccctggctccagccccggcccccgagcccaaggcgctggcgctgcaaccggtgtacccggggcccggcgccggctcctcgggtggctacttcccgcggaccgggctttcagtgcctgcggcgtcgggcgccccctacgggctactgtccgggtaccccgcgatgtacccggcgcctcagtaccaagggcacttccagctcttccgcgggctccagggacccgcgcccggtcccgccacgtccccctccttcctgagttgtttgggacccgggacggtgggcactggactcggggggactgcagaggatccaggtgtgatagccgagaccgcgccatccaagcgaggccgacgttcgtgggcgcgcaagaggcaggcagcgcacacgtgcgcgcacccgggttgcggcaagagctacaccaagagctcccacctgaaggcgcatctgcgcacgcacacaggggagaagccatacgcctgcacgtgggaaggctgcggctggagattcgcgcgctcggacgagctgacccgccactaccggaaacacacggggcagcgccccttccgctgccagctctgcccacgtgctttttcgcgctctgaccacctggccttgcacatgaagcgccacctttga

SEQ ID NO: 13 - DNA, 2286 bp, Homo sapiens
atggcccgaagaccccggcacagcatatatagcagtgacgaggatgatgaggactttgagatgtgtgaccatgactatgatgggctgcttcccaagtctggaaagcgtcacttggggaaaacaaggtggacccgggaagaggatgaaaaactgaagaagctggtggaacagaatggaacagatgactggaaagttattgccaattatctcccgaatcgaacagatgtgcagtgccagcaccgatggcagaaagtactaaaccctgagctcatcaagggtccttggaccaaagaagaagatcagagagtgatagagcttgtacagaaatacggtccgaaacgttggtctgttattgccaagcacttaaaggggagaattggaaaacaatgtagggagaggtggcataaccacttgaatccagaagttaagaaaacctcctggacagaagaggaagacagaattatttaccaggcacacaagagactggggaacagatgggcagaaatcgcaaagctactgcctggacgaactgataatgctatcaagaaccactggaattctacaatgcgtcggaaggtcgaacaggaaggttatctgcaggagtcttcaaaagccagccagccagcagtggccacaagcttccagaagaacagtcatttgatgggttttgctcaggctccgcctacagctcaactccctgccactggccagcccactgttaacaacgactattcctattaccacatttctgaagcacaaaatgtctccagtcatgttccataccctgtagcgttacatgtaaatatagtcaatgtccctcagccagctgccgcagccattcagagacactataatgatgaagaccctgagaaggaaaagcgaataaaggaattagaattgctcctaatgtcaaccgagaatgagctaaaaggacagcaggtgctaccaacacagaaccacacatgcagctaccccgggtggcacagcaccaccattgccgaccacaccagacctcatggagacagtgcacctgtttcctgtttgggagaacaccactccactccatctctgccagcggatcctggctccctacctgaagaaagcgcctcgccagcaaggtgcatgatcgtccaccagggcaccattctggataatgttaagaacctcttagaatttgcagaaacactccaatttatagattctgattcttcatcatggtgtgatctcagcagttttgaattctttgaagaagcagatttttcacctagccaacatcacacaggcaaagccctacagcttcagcaaagagagggcaatgggactaaacctgcaggagaacctagcccaagggtgaacaaacgtatgttgagtgagagttcacttgacccacccaaggtcttacctcctgcaaggcacagcacaattccactggtcatccttcgaaaaaaacggggccaggccagccccttagccactggagactgtagctccttcatatttgctgacgtcagcagttcaactcccaagcgttcccctgtcaaaagcctacccttctctccctcgcagttcttaaacacttccagtaaccatgaaaactcagacttggaaatgccttctttaacttccacccccctcattggtcacaaattgactgttacaacaccatttcatagagaccagactgtgaaaactcaaaaggaaaatactgtttttagaaccccagctatcaaaaggtcaatcttagaaagctctccaagaactcctacaccattcaaacatgcacttgcagctcaagaaattaaatacggtcccctgaagatgctacctcagacaccctctcatctagtagaagatctgcaggatgtgatcaaacaggaatctgatgaatctggaattgttgctgagtttcaagaaaatggaccacccttactgaagaaaatcaaacaagaggtggaatctccaactgataaatcaggaaacttcttctgctcacaccactgggaaggggacagtctgaatacccaactgttcacgcagacctcgcctgtggcagatgcaccgaatattcttacaagctccgttttaatggcaccagcatcagaagatgaagacaatgttctcaaagcatttacagtacctaaaaacaggtccctggcgagccccttgcagccttgtagcagtacctgggaacctgcatcctgtggaaagatggaggagcagatgacatcttccagtcaagctcgtaaatacgtgaatgcattctcagcccggacgctggtcatgtga

SEQ ID NO: 14 - DNA, 1440 bp, Homo sapiens
atggttcactccagcatgggggctccagaaataagaatgtctaagcccctggaggccgagaagcaaggtctggactccccatcagagcacacagacaccgaaagaaatggaccagacactaatcatcagaacccccaaaataagacctccccattctccgtgtccccaactggccccagtacaaagatcaaggctgaagaccccagtggcgattcagccccagcagcacccctgccccctcagccggcccagcctcatctgccccaggcccaactcatgttgacgggcagccagctagctggggacatacagcagctcctccagctccagcagctggtgcttgtgccaggccaccacctccagccacctgctcagttcctgctaccgcaggcccagcagagccagccaggcctgctaccgacaccaaatctattccagctacctcagcaaacccagggagctcttctgacctcccagccccgggccgggcttcccacacaggccgtgacccgccctacgctgcccgacccgcacctctcgcacccgcagccccccaaatgcttggagccaccatcccaccccgaggagcccagtgatctggaggagctggagcaattcgcccgcaccttcaagcaacgccgcatcaagctgggcttcacgcagggtgatgtgggcctggccatgggcaagctctacggcaacgacttcagccagacgaccatttcccgcttcgaggccctcaacctgagcttcaagaacatgtgcaaactcaagcccctcctggagaagtggctcaacgatgcagagactatgtctgtggactcaagcctgcccagccccaaccagctgagcagccccagcctgggtttcgacggcctgcccggccggagacgcaagaagaggaccagcatcgagacaaacgtccgcttcgccttagagaagagttttctagcgaaccagaagcctacctcagaggagatcctgctgatcgccgagcagctgcacatggagaaggaagtgatccgcgtctggttctgcaaccggcgccagaaggagaaacgcatcaacccctgcagtgcggcccccatgctgcccagcccagggaagccggccagctacagcccccatatggtcacaccccaagggggcgcggggaccttaccgttgtcccaagcttccagcagtctgagcacaacagttactaccttatcctcagctgtggggacgctccaccccagccggacagctggagggggtgggggcgggggcggggctgcgccccccctcaattccatcccctctgtcactcccccacccccggccaccaccaacagcacaaaccccagccctcaaggcagccactcggctatcggcttgtcaggcctgaaccccagcacgggccctggcctctggtggaaccctgccccttaccagccttga

SEQ ID NO: 15 - DNA, 4320 bp, Homo sapiens
atggccagctcggagactgagatccgctgggctgagcctggcctggggaagggcccccagcggcggcgctgggcctgggccgaggacaagagggatgtggatagaagtagttcacaaagctgggaagaagagagactctttcccaatgccaccagccccgagctcctagaggacttccgcctggcccagcagcacctgccgcccctggagtgggacccacacccgcagcccgatgggcatcaggattccgagtcaggagagacttcgggagaagaggctgaagcagaggatgtggacagcccagcaagttcccatgagcctcttgcctggctcccccagcagggccgtcagctggacatgactgaagaggagccagatgggaccctcggaagtctggaggttgaggaggctggagagagctcctcaaggttggggtatgaggctggtctcagcttggaaggccatggaaacaccagccccatggctcttgggcatggtcaggccaggggctgggtggcttctggcgaacaagccagtggggacaaactttctgaacattccgaggtcaacccatccgttgaactcagcccggcaaggtcctggagcagtgggacagtgagcctcgaccaccctagtgacagccttgattctacctgggaaggagagaccgatggcccccagcccactgccctggcagaaaccttgccagagggccccagccaccacctcctaagcccagatggcagaactggaggcagtgttgctcgggcaacccccatggaattccaggactcctcagctcccccagcccagagtccgcagcatgccacagatagatggaggagagaaacgaccagattcttctgccctcagcccaaggaacacatctggaagcagacaaagacgtcacctaagccactcccttcccgattcattggctccatcagccccctgaatccccagcccaggccaacgcggcagggcaggccgctgcccagacagggagccactctggctggccgctcctcttctaatgcccccaagtatggccgggggcagttgaactacccactccctgatttctccaaggtagggccccgggtgagattccccaaagatgagagctaccgtccccccaagtccagaagccacaacaggaagcctcaggcccctgccaggcccctcatcttcaagtctccagctgagattgtgcaggaggtgctgttgagcagtggagaagcagccctggcaaaggacacgcctcctgcccaccctatcaccagggtaccccaagaatttcagacgcctgagcaagccactgagctggtccatcagctccaggaagactaccacaggctcctcaccaagtacgctgaggccgagaacaccattgaccagctacgcctcggggccaaggtgaacctgttctctgacccaccccagcccaaccacagcatccacacgggaatggtgccccaggggaccaaggtcttgtccttcaccatcccacagccccgctctgcagagtggtggccgggcccggccgaggacccccaggcctctgcggcctcagggtggccatcagctcgaggagacttgagcccctcctcgcttaccagcatgcccaccctggggtggcttccggagaaccgggacatctctgaggaccagtcctcagcagagcagacccaggcactggcttctcaggccagccagttcctggccaaggtggagtcctttgaaagactgatacaggcaggacgtctcatgccccaggaccaagtcaagggcttccagcggctgaaggctgcccacgcggccctagaggaggagtacctgaaggcttgtcgggagcaacaccctgcccagccgcttgccggctccaaggggacgcctggaagatttgatcctcgcagggagctggaggcagagatataccgtctgggaagctgcctggaagagctgaaggaacacatagaccagacccagcaagagcctgagccgcccgggtcagactcagctctggacagcaccccagccctgccctgcctccatcagccaacgcacctgcctgctccttctggacaagcccccatgccagccatcaagacctcctgccctgagcctgctaccaccactgccgccgccagcactggcccctgcccattgcacgtaaatgtggaggtgagctctggcaacagtgaggtggaggacaggccacaggaccccctggcccgactcaggcacaaggagctgcagatggagcaagtttaccatggcctcatggagcggtacctcagtgtgaagtctctcccagaagccatgagaatggaggaggaggaagaaggagaggaggaggaggaggaagaggggggaggtgactccctggaagttgatggggtggctgcaactccagggaaagcagaggccaccagggtcctcccaaggcagtgcccggtgcaggctgagaaaagtcatggggctcccctggaggaggccacggagaagatggtatctatgaagccaccaggtttccaggcatccctggctagagacgggcacatgtcaggcctgggcaaggctgaggcagcccctccaggccctggcgtgccaccccaccctccaggcaccaagtccgcagcatcccaccaaagtagtatgaccagcctggagggaagcggcatctctgagcgccttccacagaagcctttgcaccgaggcggtgggccccacctggaggagacctggatggcgtccccagagacagacagtggctttgtgggctcagaaacaagcagagtttcacccctcacccagactccagagcaccggctctcccacatcagcacagcaggaacattagcccagccctttgctgcatctgtgcccagggatggagcttcctaccccaaggccaggggttctctgattcccagaagagccacagagcccagcacaccccggagccaagcacagaggtacctctccagcccaagtgggcctctccggcagagggcacccaacttcagcctggagcggacactggcagccgagatggcggttcctggctcagagtttgaggggcacaaacggatttctgaacagccccttcccaacaagacaatcagcccacccccagcccccgcccctgccgctgcgcctctaccctgtggaccaacagagaccatccccagcttcctgctcaccagggcagggcgagaccaggccatctgtgagctgcaagaagaggtgtcccggcttcgtctgcggctggaagacagcctgcaccagccactccagggcagcccgacacgcccagcatctgcctttgaccgccccgcccggacccgcggccggccagcagactccccagccacctggggctcccattatggcagtaaatccacagagagattgcctggtgagcctagaggtgaagagcagattgtccctccaggaaggcagcgagccaggtcttcctcagtgcctcgggaggtgctccgactgtccctgagttcagaatctgagctgccctccctaccactgttctctgagaagagcaagaccaccaaggacagtccacaggcagctcgggatggaaagagaggggtgggcagtgctggatggccagacagggtcaccttccggggccaatacacaggccacgaataccatgttctgtcccctaaggcggtcccaaaaggcaatggcacagtctcctgtccccactgccggcccattaggacccaggatgcgggtggtgctgtcacaggggacccactgggaccgcctcccgctgatacccttcagtgtcccctgtgtggtcaagttgggtctcccccagaggcagatggtccaggctcagccacctctggggcagagaaggccaccacgaggagaaaagcaccttcaactcccagccccaagcagaggagcaagcaggcggggtcgtcgccacgcccaccccccggactgtggtatctggcaacagcgcccccagcaccagcccctccagcctttgcctacatctcctcggttcccatcatgccttatccacctgccgctgtgtactatgcgcctgcaggacctacctcagcccaaccagctgccaagtggccgcccacagcctctcccccaccagcccggagacaccggcactccatccagctcgacctgggcgacctagaggagctcaacaaggccctgagccgggccgtgcaggctgccgagagcgtccgctctaccaccaggcagatgagaagctcgctgtcagccgacctgcgccaggctcacagcctgcggggctcctgcctcttctga

SEQ ID NO: 16 - DNA, 1560 bp, Homo sapiens
atggatgctgatgagggtcaagacatgtcccaagtttcagggaaggaaagcccccctgtaagcgatactccagatgagggcgatgagcccatgccgatccccgaggacctctccaccacctcgggaggacagcaaagctccaagagtgacagagtcgtggccagtaatgttaaagtagagactcagagtgatgaagagaatgggcgtgcctgtgaaatgaatggggaagaatgtgcggaggatttacgaatgcttgatgcctcgggagagaaaatgaatggctcccacagggaccaaggcagctcggctttgtcgggagttggaggcattcgacttcctaacggaaaactaaagtgtgatatctgtgggatcatttgcatcgggcccaatgtgctcatggttcacaaaagaagccacactggagaacggcccttccagtgcaatcagtgcggggcctcattcacccagaagggcaacctgctccggcacatcaagctgcattccggggagaagcccttcaaatgccacctctgcaactacgcctgccgccggagggacgccctcactggccacctgaggacgcactccgttggtaaacctcacaaatgtggatattgtggccgaagctataaacagcgaagctctttagaggaacataaagagcgctgccacaactacttggaaagcatgggccttccgggcacactgtacccagtcattaaagaagaaactaatcacagtgaaatggcagaagacctgtgcaagataggatcagagagatctctcgtgctggacagactagcaagtaacgtcgccaaacgtaagagctctatgcctcagaaatttcttggggacaagggcctgtccgacacgccctacgacagcagcgccagctacgagaaggagaacgaaatgatgaagtcccacgtgatggaccaagccatcaacaacgccatcaactacctgggggccgagtccctgcgcccgctggtgcagacgcccccgggcggttccgaggtggtcccggtcatcagcccgatgtaccagctgcacaagccgctcgcggagggcaccccgcgctccaaccactcggcccaggacagcgccgtggagaacctgctgctgctctccaaggccaagttggtgccctcggagcgcgaggcgtccccgagcaacagctgccaagactccacggacaccgagagcaacaacgaggagcagcgcagcggtctcatctacctgaccaaccacatcgccccgcacgcgcgcaacgggctgtcgctcaaggaggagcaccgcgcctacgacctgctgcgcgccgcctccgagaactcgcaggacgcgctccgcgtggtcagcaccagcggggagcagatgaaggtgtacaagtgcgaacactgccgggtgctcttcctggatcacgtcatgtacaccatccacatgggctgccacggcttccgtgatccttttgagtgcaacatgtgcggctaccacagccaggaccggtacgagttctcgtcgcacataacgcgaggggagcaccgcttccacatgagctaa

SEQ ID NO: 17 - DNA, 798 bp, Homo sapiens
atggaagggtttcccctcgtcccccctcagccatcagaagacctggtgccctatgacacggatctataccaacgccaaacgcacgagtattacccctatctcagcagtgatggggagagccatagcgaccattactgggacttccacccccaccacgtgcacagcgagttcgagagcttcgccgagaacaacttcacggagctccagagcgtgcagcccccgcagctgcagcagctctaccgccacatggagctggagcagatgcacgtcctcgatacccccatggtgccaccccatcccagtcttggccaccaggtctcctacctgccccggatgtgcctccagtacccatccctgtccccagcccagcccagctcagatgaggaggagggcgagcggcagagccccccactggaggtgtctgacggcgaggcggatggcctggagcccgggcctgggctcctgcctggggagacaggcagcaagaagaagatccgcctgtaccagttcctgttggacctgctccgcagcggcgacatgaaggacagcatctggtgggtggacaaggacaagggcaccttccagttctcgtccaagcacaaggaggcgctggcgcaccgctggggcatccagaagggcaaccgcaagaagatgacctaccagaagatggcgcgcgcgctgcgcaactacggcaagacgggcgaggtcaagaaggtgaagaagaagctcacctaccagttcagcggcgaagtgctgggccgcgggggcctggccgagcggcgccacccgccccactga

SEQ ID NO: 18 - DNA, 1497 bp, Homo sapiens
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagaggatgtcaagtggccgcccactctgcagccgcccactctgcggccgcctactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataa

SEQ ID NO: 19 - DNA, 1155 bp, Homo sapiens
atgccgcagctggactccggcgggggcggcgcgggcggcggcgacgacctcggcgcgccggacgagctgctggccttccaggatgaaggcgaggagcaggacgacaagagccgcgacagcgccgccggtcccgagcgcgacctggccgagctcaagtcgtcgctcgtgaacgagtccgagggcgcggccggcggcgcagggatcccgggggtcccgggggccggcgccggggcccgcggcgaggccgaggctctcgggcgggaacacgctgcgcagagactcttcccggacaaacttccagagcccctggaggacggcctgaaggccccggagtgcaccagcggcatgtacaaagagaccgtctactccgccttcaatctgctcatgcattacccacccccctcgggagcagggcagcacccccagccgcagcccccgctgcacaaggccaatcagcccccccacggtgtcccccaactctctctctacgaacatttcaacagcccacatcccacccctgcacctgcggacatcagccagaagcaagttcacaggcctctgcagacccctgacctctctggcttctactccctgacctcaggcagcatggggcagctcccccacactgtgagctggttcacccacccatccttgatgctaggttctggtgtacctggtcacccagcagccatcccccacccggccattgtgcccccctcagggaagcaggagctgcagcccttcgaccgcaacctgaagacacaagcagagtccaaggcagagaaggaggccaagaagccaaccatcaagaagcccctcaatgccttcatgctgtacatgaaggagatgagagccaaggtcattgcagagtgcacacttaaggagagcgctgccatcaaccagatcctgggccgcaggtggcacgcgctgtcgcgagaagagcaggccaagtactatgagctggcccgcaaggagaggcagctgcacatgcagctatacccaggctggtcagcgcgggacaactacgggaagaagaagaggcggtcgagggaaaagcaccaagaatccaccacaggaggaaaaagaaatgcattcggtacttacccggagaaggccgctgccccagccccgttccttccgatgacagtgctctag

SEQ ID NO: 20 - DNA, 798 bp, Homo sapiens; identical to SEQ ID NO: 17

SEQ ID NO: 21 - DNA, 3222 bp, Homo sapiens
atgcaccagccgcctgagtccaccgccgcggccgccgccgctgcagacattagcgctaggaagatggcgcacccggcaatgttccctcgaaggggcagcggtagtggcagcgcctctgctctcaatgcagcaggtaccggcgtcggtagtaatgccacatcttccgaggattttccgcctccgtcgctgcttcagccgccgccccctgcagcatcttctacgtcgggaccacagcctccgcctccacaaagcctgaacctcctttcgcaggctcagctgcaggcacagcctcttgcgccaggcggaactcaaatgaaaaagaaaagtggcttccagataactagcgttactcctgctcagatctccgctagtatcagctctaacaacagtatagcagaggacactgagagctatgatgatctggatgaatctcacacggaagatctctcttcttcggagatccttgatgtgtcactttccagggctactgacttaggggagcccgaacgcagctcctcagaagagaccctaaataacttccaggaagccgagacacctggggcagtctctcccaaccagccccaccttcctcagcctcatttgcctcaccttccacaacagaatgttgtgatcaatgggaatgctcatccacaccacctccatcaccaccatcagattcatcatgggcaccacctccaacatggtcaccaccatccatctcatgttgctgtggccagtgcatccattactggtgggccaccctcaagcccagtatctagaaaactctctacaactggaagctctgacagtatcacaccagttgcaccaacttctgctgtatcatccagtggttcacctgcatctgtaatgactaatatgcgtgctccaagtactacaggtggaataggtataaattctgttactggcactagtacagtaaataatgttaacattactgctgtgggtagttttaatcctaatgtgacaagcagcatgcttggtaatgttaatataagtacaagcaatattcctagtgctgctggtgtgagtgttgggcctggagttaccagtggtgttaatgtgaatatcttgagtggcatgggcaatggtactatttcttcctctgctgctgttagcagtgttcctaatgcagctgcagggatgactgggggatcggtttcaagtcagcagcaacaaccaacagttaacacttcgaggttcagagttgtgaagttagattctagttctgagccctttaaaaaaggtagatggacttgcactgagttctatgaaaaagaaaatgctgtacctgctacagaaggtgtgctgataaataaagtggtggagactgtaaagcaaaatccgatagaagtgacttctgaaagggagagcactagtgggagttcagtgagcagtagtgtcagcacactgagtcactatacagagagtgtgggaagtggagagatgggagcccctactgtggtggtgcagcagcagcagcagcaacaacaacaacaacagcaacaaccagctctccaaggtgtgaccctccaacagatggattttggtagcactggtccacagagtattccagcagttagtataccacagagtatttctcagtcacagatctcacaagtacaattacagtctcaagaactgagctatcagcaaaagcaaggtcttcagccagtacctctgcaagccactatgagtgctgcaactggtatccagccatcgcctgtaaatgtggttggtgtaacttcagctttaggtcagcagccttccatttccagtttggctcaaccccagctaccatattctcaggcggctcctccagtgcaaactccccttccaggggcaccaccaccccaacagttacagtatggacaacagcaaccaatggtttctacacagatggccccaggccatgtcaaatcagtgactcaaaatcctgcttcagagtatgtacaacagcagccaattcttcaaacagcaatgtcctccggacagcccagttctgcaggagtaggagcaggaacaacagtgattcctgtggctcagccacagggtatccagctgccagtgcagcccacagcagtcccagcacaacctgcaggggcatctgtccagcctgttggccaggctccggcagcagtgtctgctgtacctactggcagtcagattgcaaatattggtcagcaagcaaacatacctactgcagtgcagcagccctctacccaggttccaccttcagttattcagcagggtgctcctccatcttcgcaagtggttccacctgctcaaactgggattattcatcagggagttcaaactagtgctccaagccttcctcaacaattggttattgcatcccaaagttccttgttaactgtgcctccccagccacaaggagtagaaccagtagctcaaggaattgtttcacagcagttgcctgcagttagttctttgccctctgctagtagtatttctgttacaagtcaggttagttcaactggtccttctggaatgccttctgccccaacaaacttggttccaccacaaaatatagcacaaacccctgctacccaaaatggtaatttggttcaaagtgttagtcaacctcccttgatagcaactaatacaaatttgcctttggcacaacagataccactaagttctacccagttctccgcacaatcattagctcaggcaattggaagccaaattgaagatgccaggcgtgcagcggagccctccttagttggcttacctcagactatcagtggtgacagtgggggaatgtcagcagtttcagatgggagtagcagcagcctagcagcctctgcttctcttttcccgttgaaggtgctaccgctgacgacacccctggtggatggcgaggatgagagctcctctggtgcaagtgtggtagctattgacaacaaaatcgagcaagctatggatctagtgaaaagccatttgatgtatgcggtcagagaagaagtggaggtcctcaaagagcaaatcaaagaactaatagagaaaaattcccagctggagcaggagaacaatctgctgaagacactggccagtcctgagcagcttgcccagtttcaggcccagctgcagactggctccccccctgccaccacccagccacagggcaccacacagccccccgcccagccagcatcgcagggctcaggaccaaccgcatag

SEQ ID NO: 22 - DNA, 873 bp, Homo sapiens
atgggcgatgggggcgccgagcgcgaccggggccccgcgcgccgggcggagtctggtggcggcggtgggcgctgcggagaccgcagcggagcgggggacttgcgagctgatggcggtggccacagcccaacggaggtggccgggacctcagcctccagtcccgcaggctccagggagagtggagccgacagcgacgggcagcccgggcccggcgaggcagaccactgccgccgcatactggtgcgagatgccaaagggacaattcgggaaattgtcctgcctaagggcctggacctggaccggcccaagcggacacgtacatccttcactgccgagcagctgtaccgcctggagatggagttccagcgctgccagtatgtggtgggccgcgagcgcactgagctggcccgccagctgaacctctccgagacccaggtgaaggtctggttccagaaccgccgcaccaagcagaagaaagaccagagcagagacctggagaagcgggcgtcctcctcagcctccgaggcctttgccacctccaacattctgcggctgctggagcagggccggctgctctctgtgcccagggcccctagcctcctggcgctgacccctagcctgccaggcctacctgccagccacaggggcacctccttaggtgaccccaggaactcctccccacgcctcaacccgctgtcctcggcctcagcgtcccccccactgccgccccctctgccagctgtctgcttttcctcggccccgctcctggatctgcctgccggctacgaactgggttcctcggccttcgagccatacagctggctagaacggaaagtgggcagcgccagcagctgcaagaaagctaacacttaa

SEQ ID NO: 23 - DNA, 1869 bp, Homo sapiens
atgtccatgaggagccccatctctgcccagctggccctggatggcgttggcaccatggtgaactgcaccatcaagtcagaggagaagaaagagccttgccacgaggccccccagggctcagccactgccgctgaacctcagcctggagacccagcccgggcctcccaggatagtgctgacccccaagctccagcccaggggaatttcaggggctcctgggactgtagctctccagagggtaatgggtccccagaacccaagagaccaggagtgtcggaggctgcctctggaagccaggagaagctggacttcaaccgaaatttgaaagaagtggtgccagccatagagaagctgttgtccagtgactggaaggagaggtttctaggaaggaactctatggaagccaaagatgtcaaagggacccaagagagcctagcagagaaggagctccagcttctggtcatgattcaccagctgtccaccctgcgggaccagctcctgacagcccactcggagcagaagaacatggctgccatgctgtttgagaagcagcagcagcagatggagcttgcccggcagcagcaggagcagattgcaaagcagcagcagcagctgattcagcagcagcataagatcaacctccttcagcagcagatccagcaggttaacatgccttatgtcatgatcccagccttccccccaagccaccaacctctgcctgtcacccctgactcccagctggccttacccattcagcccattccctgcaaaccagtggagtatccgctgcagctgctgcacagcccccctgccccagtggtgaagaggcctggggccatggccacccaccaccccctgcaggagccctcccagcccctgaacctcacagccaagcccaaggcccccgagctgcccaacacctccagctccccaagcctgaagatgagcagctgtgtgccccgcccccccagccatggaggccccacgcgggacctgcagtccagccccccgagcctgcctctgggcttccttggtgaaggggacgctgtcaccaaagccatccaggatgctcggcagctgctgcacagccacagtggggccttggatggctcccccaacacccccttccgtaaggacctcatcagcctggactcatccccagccaaggagcggctggaggacggctgtgtgcacccactggaggaagccatgctgagctgcgacatggatggctcccgccacttccccgagtcccgaaacagcagccacatcaagaggcccatgaacgccttcatggtgtgggccaaggatgagcggaggaagatcctgcaagccttcccagacatgcacaactccagcatcagcaagatccttggatctcgctggaagtccatgaccaaccaggagaagcagccctactatgaggaacaggcgcggctgagccggcagcacctggagaagtatcctgactacaagtacaagccgcggcccaagcgcacctgcatcgtggagggcaagcggctgcgcgtgggagagtacaaggccctgatgaggacccggcgtcaggatgcccgccagagctacgtgatccccccgcaggctggccaggtgcagatgagctcctcagatgtcctgtaccctcgggcagcaggcatgccgctggcacagccactggtggagcactatgtccctcgtagcctggaccccaacatgcctgtgatcgtcaacacctgcagcctcagagaggagggtgagggcacagatgacaggcactcggtggctgatggcgagatgtaccggtacagcgaggacgaggactcggagggcgaagagaagagcgatggggagttggtggtgctcacagactga

SEQ ID NO: 24 - DNA, 2154 bp, Homo sapiens
atggcaaccccggcggcggtcaaccctccggaaatggcttcagacatacctggatctgtgacgttgcccgttgcccccatggcggccaccggacaggtgaggatggcgggggccatgcctgcccgtggaggaaagcggcgttccggaatggacttcgatgatgaagatggtgaaggccccagtaaattttcaagagagaatcatagtgaaatcgaaaggcgcagacggaacaagatgactcagtacatcacggagctctccgacatggtccccacatgcagcgcactggctcggaagccagacaagctcaccatcctccgcatggccgtctcgcacatgaagtccatgaggggtacagggaacaagtccaccgatggcgcgtacaagccttccttcctcacagagcaggaactgaagcatctcatccttgaagcagctgatggatttctgtttgtggtggctgctgagacagggcgagtgatttatgtgtctgactccgtcacccctgttctgaaccagccccagtcagagtggtttgggagcacactgtatgaacaggtgcatcctgatgacgtggagaagctgagagagcaactgtgcacctcagaaaactcaatgacaggccggatcttggacctgaagactgggacggtcaagaaagaagggcagcagtcatccatgaggatgtgcatgggctcgcggcggtctttcatctgcaggatgaggtgtggaaatgctcctttggaccaccttcctctaaacagaataaccaccatgaggaaaaggttcaggaatggccttggccctgtgaaagaaggagaagcccaatatgctgtggtccactgtacaggatacatcaaggcctggccaccagcaggaatgaccatacctgaagaagacgctgatgtgggacaaggcagtaaatattgcctcgtggcaattgggagactccaggtgaccagctctcctgtatgcatggacatgaatgggatgtcggtgcccacagagttcttatcccggcataactccgatggaatcatcacatttgtggatccaagatgtatcagtgtgattggctaccaaccccaggatcttctgggaaaggacattttggaattctgccaccctgaggatcaaagccatctgcgtgagagcttccagcaggtggttaagctgaaaggccaagtcctgtcggtcatgtatcgatttcgcaccaagaaccgggagtggatgttgatccgcaccagcagcttcacattccagaatccctattctgatgagattgagtacatcatctgcaccaacaccaacgtcaagcaacttcagcaacagcaggcagaattggaagtgcaccagagagatggattgtcatcgtatgacttatcccaggtccccgtccccaacctaccagccggtgttcatgaggccgggaagtccgtggaaaaggcggatgcaatcttctcccaggaaagagatcctcggtttgctgaaatgtttgcaggaattagtgcatcggagaagaagatgatgagctcagcctctgcagcaggaacccagcagatctactcccaaggaagcccatttccctctggacactccgggaaggccttcagctcttcagtggttcatgtgcctggagtgaatgatattcagtcctcttcttccacgggccagaacatgtcccaaatctcccggcagctaaaccagagtcaggtggcatggacagggagtcgtccgccctttccgggacagcaaatcccatctcagtccagcaagactcagtcatctccctttgggattggaacgagccacacctacccggcagacccctcttcctacagccccctctccagcccagctacctcctcgccaagtgggaatgcctactccagtcttgccaacaggactccagggttcgctgaaagtggacaaagtagcgggcagttccaagggcggccctcggaagtctggtcgcagtggcaaagccagcaccatggccagcagagcggtgagcagcactcccaccagcagcccggtcagactgaagtgttccaggacatgctgcccatgccaggagatccaacccaggggactggcaactataacatcgaagactttgccgacctgggcatgtttccaccgttttctgagtag

SEQ ID NO: 25 - DNA, 1434 bp, Homo sapiens
atgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactag

SEQ ID NO: 26 - DNA, 3300 bp, Homo sapiens
atggcacaccttgggcccaccccacctccacatagccttaattacaaatcagaggacaggcttagtgagcaagactggccagcatatttcaaggtcccatgttgtggggttgatacatctcaaattgagtcagaagaggcagaagtggatgtgagagaaagagagacacagagagacagagagccaaagagggcaagagacttgactttaagagactcctgtactgacaactccatgcagttcggaaccagaacgactacggctgaaccagggttcatggggacatggcaaaacgctgatactaacctcttattcagaatgtcccaacaggccatccgttgcacactggtaaactgcacatgtgaatgttttcagccagggaagattaacctgaggacttgtgatcagtgtaaacatggctgggtggcacatgccttggataagctcagcacgcagcacctgtaccaccccacccaagtggagattgtgcagtccaacgtcgtgtttgacatcagcagcctgatgctctatgggacacaagcagtgcctgtgcggctaaagatcctgctggaccgtctcttcagcgtcctgaagcaagaggaggtactgcacatactgcacggccttggctggactctgcgggactatgtccgaggatacatccttcaggatgctgctggcaaggtgctggaccgctgggccatcatgtctcgagaagaggaaatcatcacccttcagcagtttctgcggtttggagaaaccaaatccattgtggagctgatggcaattcaggagaaagaagggcaggccgtggctgtaccatcttcaaagacagactcagatataaggactttcattgagagcaataatcgcaccaggagtcccagcctccttgctcacttagagaacagcaatccttccagcattcatcacttcgaaaacatcccaaacagccttgcatttctgcttccattccagtacataaaccctgtctcagcaccactgctagggttgcctccaaatgggctactgttagagcaaccagggttgaggctgcgggaacccagcctttcaactcagaatgaatataatgagagcagcgaatccgaagtttctcccacaccttataagaatgatcaaacacccaatagaaatgccctgaccagcattactaatgtggagcccaaaaccgagccagcctgtgtctctcccattcagaattctgccccagtcagtgatctaaccaaaactgaacacccaaaaagctcattccggattcatcggatgagaaggatggggtcagcctctaggaaaggaagagtgttctgtaatgcatgtgggaagacattctatgacaaaggtactctcaaaattcattacaatgctgttcacctgaagatcaaacatcgatgcaccattgaaggttgcaacatggtctttagctccctccgaagtcgtaatcgccacagtgcaaaccccaatcctcgccttcacatgcctatgctaaggaataaccgagataaagatttaattcgggccacctcaggagctgccacccctgtcatagcaagtacaaaatcaaatctggcactcacaagccctggccgacccccaatgggttttaccactccccctctagaccctgtcttgcaaaatcctctccctagccagctagtattttctgggctaaagactgtacaaccagttcctccattttatagaagtttactcactccaggggaaatggtgagtcctccaacctccctcccaaccagtcccatcattccaaccagtggtaccatagagcagcaccccccgccaccctctgagccagtagtgccagcagtgatgatggccacccatgagcccagtgctgacctggcacccaagaaaaagcccaggaagtcaagcatgcctgtgaagattgagaaggaaattattgataccgccgatgagtttgatgatgaagatgatgaccccaatgatggtggagctgtggtcaatgacatgagccatgacaatcattgtcactcccaagaggagatgagcccaggcatgtctgtgaaggacttttctaagcataacaggacccggtgcatttcaaggactgaaataaggagggccgacagcatgacttctgaagaccaagaacctgagcgggactatgagaacgagtctgagtcttcggagcccaaactgggcgaggaatccatggaaggggatgagcacattcacagcgaagtgagtgaaaaagtcctgatgaatagtgagaggcctgatgagaaccacagtgagccctctcaccaggacgtcatcaaggtgaaggaagaatttacagaccccacttacgacatgttttacatgagccagtatggactgtacaatggtgggggtgccagcatggccgccttgcatgagagctttacatcgtctctgaattatggcagccctcaaaagttctccccagaaggtgacctatgttctagcccagaccccaaaatctgttatgtgtgcaagaagagtttcaaaagctcctacagtgtgaaacttcactacaggaacgttcacttgaaagagatgcacgtctgcacagtggctggttgcaatgctgcattcccctctcgccgaagccgagacagacacagtgccaacataaacctacatcgtaaactgttgaccaaagaactcgatgacatgggcctggactcgtcgcagccctcccttagcaaggacctccgcgatgaatttttggtgaagatatatggtgcccagcaccccatggggctcgatgtcagggaagacgcctcctctcccgcagggactgaagactcccacctgaacgggtatgggagaggcatggcagaggactacatggtccttgacttgagcaccacctccagcctccagtccagcagcagtatccattcctccagagaatccgacgcaggcagcgatgaggggattcttctcgatgacattgacggggcgagtgacagtggggagtcggcacacaaggccgaggcccctgccctccctggcagcctaggggctgaagtttcaggatctcttatgttcagcagcttgtctgggagcaatggtgggatcatgtgcaacatttgccacaaaatgtacagcaacaaggggaccctgagagtgcactacaaaactgtgcatttgagagaaatgcacaagtgcaaagtcccaggttgcaatatgatgttttcctctgtacgaagccgaaatcggcacagtcagaaccctaatctccacaaaaacattcccttcacttcagtagattag

SEQ ID NO: 27 - DNA, 873 bp, Homo sapiens
atgagttcgtacttcgtgaacccgctgtactccaagtacaaggcggcggctgcggcggcggcggcggcgggcgaggccatcaatcccacttactacgactgtcacttcgcgcccgaggtcggcggccgtcacgccgccgccgcagcagccctgcagctctatggcaacagcgccgccggcttcccgcacgcgcccccgcaggcgcacgcgcacccgcacccgtccccgccgccctccgggactgggtgcggcggtagggaaggccggggccaggagtacttccaccccggcgggggcagcccggccgctgcctaccaggccgccccccctcctcctccgcatcctccgcctccgccgccacctcccccctgcggcgggattgcctgtcacggggagcccgcgaagttttacggatacgataacttacagagacagccgatttttacgacccagcaagaggccgagctggtacaatatcctgactgtaaatcgtccagtggtaatattggcgaggacccagaccacttaaatcagagctcgtctccttctcaaatgtttccgtggatgagaccacaagcagctcctggtagacgaagaggaagacaaacctacagtcgcttccaaactctagagttggaaaaggaatttctttttaacccctatctgaccaggaaaagaagaatcgaggtttcccacgccctagccctcaccgagagacaggtaaaaatctggttccagaacaggagaatgaaatggaaaaaggaaaacaacaaggacaaatttcccgtttcccggcaggaggtgaaggacggggaaacgaaaaaggaagcccaagagctggaggaagacagagccgaaggcctgacaaattaa

SEQ ID NO: 28 - DNA, 2793 bp, Homo sapiens
atgaatggaagatcatgcagcatgagtctccaccggacatcgggaaccccacaggggcctaggatggtcagtggtcatcacattcctgccatccgagcccactccgggactcctggcccctcgccctgtggcagcacatcgagtcccactatggcaagccttgctaacaacctccatctcaagatgccctcaggaggagggatggctcctcagaacaacgtggctgagagccgcatccatctgcctgccttaagccccaggagacaaatgctcaccaatgggaagccgcgattccaggtcacccaggctggaggcatgtcagggtcacatactttaaagccaaagcagcaggagtttggaagcccttttcctccaaatcctgggaaaggggctcttggctttgggcctcagtgcaagtccattggaaaaggcagctgcaacaatctagtggtcaccagcagtcccatgatggttcagcgactgggactcatttcacctccagcaagccaggtctctacagcatgcaaccagatcagtcctagcttacagagggcaatgaatgcagccaacctgaatatacctccttcagataccaggtcccttatttcgcgtgagtctttggcgtccacgaccttgagtctgacggaaagtcagtcggcctcaagcatgaagcaggagtggtcccagggctacagggccctcccttcgctctccaaccacggctctcagaatggccttgatctaggggatctccttagccttcctcccgggacatccatgtccagcaatagtgtctctaactcattaccatcctacctttttggcacggaaagtagccactctccttaccctagtcctcggcactcatccaccaggtcccactcggcccgctccaagaagagagcgctgtccttgtccccgctgtccgatggcatcgggatagatttcaataccatcatccgcacgtcgcccacgtccttggtggcctacatcaacgggtcgagggcttcgccggccaacctgtccccgcagccggaggtctacgggcatttcctgggcgtgcgcggcagctgcattccccagccgcgcccggtgcccggcagccagaagggcgtgctggtggcccctggaggcctggcgctgccggcctacggcgaggacggggccctggagcacgagcgcatgcaacagctggagcacggcggcctgcagccaggcctggtcaaccacatggtggtgcagcatggcctgccgggccccgacagccagtcggccggcctgttcaagaccgaacgcctggaggagttcccgggcagcaccgtagacctaccccccgcgcctccgctccctcctctgccgccgcccccaggccccccacccccttaccatgcccatgcgcaccttcaccacccggagctcgggccccacgcccagcagctggccttgccccaggccaccctggacgacgacggggagatggacggcatcgggggcaagcattgctgccgctggatcgactgcagcgccctgtacgaccagcaggaggagctcgtgcggcacatcgagaaggtccacatcgaccagcgcaaaggggaggacttcacttgcttctgggccggttgccctcgaagatacaagcccttcaacgcccgctataaactgctgatccacatgagagtccactctggggagaagcccaacaagtgtacgtttgaaggttgcgagaaggccttttcaaggcttgaaaatctcaagatccacttgcggagccacacaggcgagaagccgtatttgtgccagcatccgggttgtcagaaggccttcagtaactccagtgaccgcgccaaacaccagcggacgcatctggacaccaaaccttatgcttgtcaaattccaggatgtaccaaacgctacacagacccaagttccctaagaaagcatgtgaaggcacattcttccaaagagcaacaagcaaggaaaaagttgcggtccagcacagagctccatccagacctgctcacagattgcctcaccgtgcagtccctgcagccggccacttcccctagagatgctgctgctgaagggaccgtgggacgctcccctggacccgggcctgacctctattcagctcccattttctccagcaattattcaagccgaagtggaacagctgctggggccgtaccacccccacatcctgtcagtcacccttctccaggacataatgtacaggggagccctcacaacccctcctcccagttacctccactcacagctgtggacgcaggagctgagaggtttgcaccttctgctccatctcctcaccacatcagcccccggagagttccagctccttcttcaatactgcaaagaacacagcctccctatacccagcagccatcaggttcacacctgaagtcctatcagccagaaacaaactcttcttttcaaccaaatggtatccatgtccatggattttatgggcagctgcagaagttctgtcccccacactaccccgattcccagagaattgtgccgcctgtcagctcctgcagtgtggtgccttcgtttgaggactgcctagtccctacatccatgggccaggccagttttgatgttttccacagagccttctcgactcactcgggcattacagtgtatgatttaccttcaagttcctcgagcctctttggggagtctctccgcagcggggctgaagatgctaccttcttgcagatcagcaccgtggaccgctgtcctagccagctctcctctgtctacaccgaaggctaa

SEQ ID NO: 29 - DNA, 1398 bp, Homo sapiens
atgaccctgagcactgagatgtccgatgcctctggcctcgccgaggaaacagacatcgacgtggtgggggagggcgaggacgaagaagacgaggaagaggaggacgacgacgagggcggcggtggcgggccccggctggctgtccccgcgcagcggcggcggcggcggcgctcgtacgccggggaggacgagctggaggatctggaggaggaggaggacgacgatgacatcctgctggccccgcctgctgggggctccccggcgcccccgggcccggccccggcggcgggggcaggagccggtgggggcggcggcggcggcggcgcgggcggcggcgggagcgcgggtagcggcgccaagaacccgctggtgaagccgccctactcgtatatcgcgctcatcactatggccatcctgcagagccccaagaagcggctgacgctgagcgagatctgtgagttcatcagcggccgcttcccctactaccgggagaagttccccgcctggcagaacagcatccgccacaacctctcgctcaacgactgcttcgtcaagatcccccgcgagcccggcaacccgggcaagggcaactactggacgctggacccggagtccgccgacatgttcgacaacggcagcttcctgcgccggaggaagcgcttcaagcggcagccgctgctcccacccaacgccgcggccgccgagtctctgctgctgcgcggcgcgggagccgcagggggcgcgggcgacccggcagccgccgccgcgctcttcccgcccgcgcccccgccgcccccgcatgcctacggctacggcccctacggctgcggctacggcctgcagctgccgccttacgcgccgccctcggccctcttcgccgccgcagcggccgccgccgccgccgccgccttccacccgcactcgcccccgccgcccccgccaccgcacggcgcggccgccgagctggcccggaccgccttcggctaccggccgcacccgctcggcgccgccctacccggccccctgccggcctccgcggccaaggcgggcggcccgggcgcctcagcgctggcgcgctcgcccttctccatcgagagcatcatcgggggcagcttgggcccggccgccgctgccgccgccgccgcgcaggccgccgccgccgctcaggcctcgccctcgccctcgccggtggcggcgccgccagctcccggatccagcggaggaggctgcgcggcgcaggcggccgtgggcccggcggccgcgctcacccgatccctcgtggccgccgcggccgccgccgcctcctcagtctcctcgtccgccgccttggggactctgcaccaagggactgccctgtccagtgtcgagaactttactgctaggatttccaattgttaa

SEQ ID NO: 30 - DNA, 1365 bp, Homo sapiens
atggccaccaataaggagcgactctttgcggctggtgccctggggcctggatctggctacccaggggcaggtttccccttcgccttcccaggggcactcagggggtctccgcctttcgagatgctgagccctagcttccggggcctgggccagcctgacctccccaaggagatggcctctctgtcggtggagacacagagcaccagctcagaggagatggtgcccagctcgccctcgccccctccgcctcctcgggtctacaagccatgcttcgtgtgcaatgacaagtcctctggctaccactatggggtcagctcttgtgaaggctgcaagggcttctttcgccgaagcatccagaagaacatggtgtacacgtgtcaccgcgacaaaaactgtatcatcaacaaggtgaccaggaatcgctgccagtactgccggctacagaagtgcttcgaagtgggcatgtccaaggaagctgtgcgaaatgaccggaacaagaagaagaaagaggtgaaggaagaagggtcacctgacagctatgagctgagccctcagttagaagagctcatcaccaaggtcagcaaagcccatcaggagactttcccctcgctctgccagctgggcaagtataccacgaactccagtgcagaccaccgcgtgcagctggatctggggctgtgggacaagttcagtgagctggctaccaagtgcatcatcaagatcgtggagtttgccaagcggttgcctggctttacagggctcagcattgctgaccagatcactctgctcaaagctgcctgcctagatatcctgatgctgcgtatctgcacaaggtacaccccagagcaggacaccatgaccttctccgacgggctgaccctgaaccggacccagatgcacaatgccggcttcgggcccctcacagaccttgtctttgcctttgctgggcagctcctgcccctggagatggatgacaccgagacagggctgctcagcgccatctgcctcatctgcggagaccgcatggacctggaggagcccgaaaaagtggacaagctgcaggagccactgctggaagccctgaggctgtacgcccggcgccggcggcccagccagccctacatgttcccaaggatgctaatgaaaatcaccgacctccggggcatcagcactaagggagctgaaagggccattactctgaagatggagattccaggcccgatgcctcccttaatccgagagatgctggagaaccctgaaatgtttgaggatgactcctcgcagcctggtccccaccccaatgcctctagcgaggatgaggttcctgggggccagggcaaagggggcctgaagtccccagcctga

SEQ ID NO: 31 - DNA, 1266 bp, Homo sapiens
atggcccggaggtatgatgagctgccgcactacccaggcatcgtggatggccccgcagccctggctagcttcccagagacagtgcccgcagtaccagggccctatggcccgcaccggcctccccagcccctgcccccaggcttggacagcgacggcctgaagagggagaaggatgagatctatggacacccgctcttccccctcttggccctggtctttgagaaatgtgaactggctacatgctctccccgtgacggggccggagctgggctggggacaccccctggaggtgacgtctgctcctctgattccttcaacgaggacatcgctgcctttgccaagcaggttcgctctgagaggcccctcttctcctccaacccagaactggacaatctgatgatccaggccatccaggtgctgcggttccacctgctggagctggagaaggtccacgacctgtgcgacaacttctgtcaccgctacatcacctgcctcaagggaaagatgcccatcgacctggtcatcgaggatcgggacggcggctgcagggaggacttcgaggactacccagcctcctgccccagcctcccagaccagaataatatgtggattcgagaccatgaggatagtgggtctgtacatttggggaccccaggtccatccagtgggggcctggcctcccagagtggggacaactccagtgaccaaggagacgggctggacaccagcgtggcctctcccagttctggtggagaagatgaggacttggaccaggagcgacggcgaaacaagaagagggggatcttccccaaggtggccaccaacatcatgcgagcctggttgttccagcacctctcgagacgctcagaagcgccggttctcccagacgtctgcctgggcctgggctccccatcccccggaccccggtgggccagaccttggggttcagactgcggccggccaggcaggcagagtgactcttgctggtggctgcagcacccgtacccctcggaggagcagaagaaacagctggcgcaggacacggggctcaccatcctgcaagtcaacaactggttcattaacgcccggagacgcatcgtgcaacctatgatcgatcaatccaaccgcacagggcagggtgcagccttcagcccagagggccagcccatcgggggctataccgagacgcagccacacgtggccgtccggcctccgggatcagtggggatgagtttgaacttggaaggagaatggcattatctatag

SEQ ID NO: 32 - DNA, 1386 bp, Homo sapiens
atggaggacctggatgccctgctctctgacctggagactaccacctcgcacatgccaaggtcaggggctcccaaagagcgccctgcggagcctctcacccctcccccatcctatggccaccagccacagacagggtctggggagtcttcaggagcctcgggggacaaggaccacctgtacagcacggtatgcaagcctcggtccccaaagcctgcagccccggcggcccctccattctcctcttccagcggtgtcttgggtaccgggctctgtgagctagatcggttgcttcaggaacttaatgccactcagttcaacatcacagatgaaatcatgtctcagttcccatctagcaaggtggcttcaggagagcagaaggaggaccagtctgaagataagaaaagacccagcctcccttccagcccgtctcctggcctcccaaaggcttctgccacctcagccactctggagctggatagactgatggcctcactctctgacttccgcgttcaaaaccatcttccagcctctgggccaactcagccaccggtggtgagctccacaaatgagggctccccatccccaccagagccgactggcaagggcagcctagacaccatgctggggctgctgcagtccgacctcagccgccggggtgttcccacccaggccaaaggcctctgtggctcctgcaataaacctattgctgggcaagtggtgacggctctgggccgcgcctggcaccccgagcacttcgtttgcggaggctgttccaccgccctgggaggcagcagcttcttcgagaaggatggagcccccttctgccccgagtgctactttgagcgcttctcgccaagatgtggcttctgcaaccagcccatccgacacaagatggtgaccgccttgggcactcactggcacccagagcatttctgctgcgtcagttgcggggagcccttcggagatgagggtttccacgagcgcgagggccgcccctactgccgccgggacttcctgcagctgttcgccccgcgctgccagggctgccagggccccatcctggataactacatctcggcgctcagcgcgctctggcacccggactgtttcgtctgcagggaatgcttcgcgcccttctcgggaggcagctttttcgagcacgagggccgcccgttgtgcgagaaccacttccacgcacgacgcggctcgctgtgcgccacgtgtggcctccctgtgaccggccgctgcgtgtcggccctgggtcgccgcttccacccggaccacttcacatgcaccttctgcctgcgcccgctcaccaaggggtccttccaggagcgcgccggcaagccctactgccagccctgcttcctgaagctcttcggctga

SEQ ID NO: 33 - DNA, 2172 bp, Homo sapiens
atgagcctctccatgagagatccggtcattcctgggacaagcatggcctaccatccgttcctacctcaccgggcgccggacttcgccatgagcgcggtgctgggtcaccagccgccgttcttccccgcgctgacgctgcctcccaacggcgcggcggcgctctcgctgccgggcgccctggccaagccgatcatggatcaattggtgggggcggccgagaccggcatcccgttctcctccctggggccccaggcgcatctgaggcctttgaagaccatggagcccgaagaagaggtggaggacgaccccaaggtgcacctggaggctaaagaactttgggatcagtttcacaagcggggcaccgagatggtcattaccaagtcgggaaggcgaatgtttcctccatttaaagtgagatgttctgggctggataaaaaagccaaatacattttattgatggacattatagctgctgatgactgtcgttataaatttcacaattctcggtggatggtggctggtaaggccgaccccgaaatgccaaagaggatgtacattcacccggacagccccgctactggggaacagtggatgtccaaagtcgtcactttccacaaactgaaactcaccaacaacatttcagacaaacatggatttactatattgaactccatgcacaaataccagccccggttccacattgtaagagccaatgacatcttgaaactcccttatagtacatttcggacatacttgttccccgaaactgaattcatcgctgtgactgcataccagaatgataagataacccagttaaaaatagacaacaacccttttgcaaaaggtttccgggacactggaaatggccgaagagaaaaaagaaaacagctcaccctgcagtccatgagggtgtttgatgaaagacacaaaaaggagaatgggacctctgatgagtcctccagtgaacaagcagctttcaactgcttcgcccaggcttcttctccagccgcctccactgtagggacatcgaacctcaaagatttatgtcccagcgagggtgagagcgacgccgaggccgagagcaaagaggagcatggccccgaggcctgcgacgcggccaagatctccaccaccacgtcggaggagccctgccgtgacaagggcagccccgcggtcaaggctcaccttttcgctgctgagcggccccgggacagcgggcggctggacaaagcgtcgcccgactcacgccatagccccgccaccatctcgtccagcactcgcggcctgggcgcggaggagcgcaggagcccggttcgcgagggcacagcgccggccaaggtggaagaggcgcgcgcgctcccgggcaaggaggccttcgcgccgctcacggtgcagacggacgcggccgccgcgcacctggcccagggccccctgcctggcctcggcttcgccccgggcctggcgggccaacagttcttcaacgggcacccgctcttcctgcaccccagccagtttgccatggggggcgccttctccagcatggcggccgctggcatgggtcccctcctggccacggtttctggggcctccaccggtgtctcgggcctggattccacggccatggcctctgccgctgcggcgcagggactgtccggggcgtccgcggccaccctgcccttccacctccagcagcacgtcctggcctctcagggcctggccatgtcccctttcggaagcctgttcccttacccctacacgtacatggccgcagcggcggccgcctcctctgcggcagcctccagctcggtgcaccgccaccccttcctcaatctgaacaccatgcgcccgcggctgcgctacagcccctactccatcccggtgccggtcccggacggcagcagtctgctcaccaccgccctgccctccatggcggcggccgcggggcccctggacggcaaagtcgccgccctggccgccagcccggcctcggtggcagtggactcgggctctgaactcaacagccgctcctccacgctctcctccagctccatgtccttgtcgcccaaactctgcgcggagaaagaggcggccaccagcgaactgcagagcatccagcggttggttagcggcttggaagccaagccggacaggtcccgcagcgcgtccccgtag

SEQ ID NO: 34 - DNA, 1530 bp, Homo sapiens
atgaatctcctggaccccttcatgaagatgaccgacgagcaggagaagggcctgtccggcgcccccagccccaccatgtccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggcggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggaggccacggagcagacgcacatctcccccaacgccatcttcaaggcgctgcaggccgactcgccacactcctcctccggcatgagcgaggtgcactcccccggcgagcactcggggcaatcccagggcccaccgaccccacccaccacccccaaaaccgacgtgcagccgggcaaggctgacctgaagcgagaggggcgccccttgccagaggggggcagacagccccctatcgacttccgcgacgtggacatcggcgagctgagcagcgacgtcatctccaacatcgagaccttcgatgtcaacgagtttgaccagtacctgccgcccaacggccacccgggggtgccggccacgcacggccaggtcacctacacgggcagctacggcatcagcagcaccgcggccaccccggcgagcgcgggccacgtgtggatgtccaagcagcaggcgccgccgccacccccgcagcagcccccacaggccccgccggccccgcaggcgcccccgcagccgcaggcggcgcccccacagcagccggcggcacccccgcagcagccacaggcgcacacgctgaccacgctgagcagcgagccgggccagtcccagcgaacgcacatcaagacggagcagctgagccccagccactacagcgagcagcagcagcactcgccccaacagatcgcctacagccccttcaacctcccacactacagcccctcctacccgcccatcacccgctcacagtacgactacaccgaccaccagaactccagctcctactacagccacgcggcaggccagggcaccggcctctactccaccttcacctacatgaaccccgctcagcgccccatgtacacccccatcgccgacacctctggggtcccttccatcccgcagacccacagcccccagcactgggaacaacccgtctacacacagctcactcgaccttga

SEQ ID NO: 35 - DNA, 2613 bp, Homo sapiens
atgacagctgacaaggagaagaaaaggagtagctcggagaggaggaaggagaagtcccgggatgctgcgcggtgccggcggagcaaggagacggaggtgttctatgagctggcccatgagctgcctctgccccacagtgtgagctcccatctggacaaggcctccatcatgcgactggcaatcagcttcctgcgaacacacaagctcctctcctcagtttgctctgaaaacgagtccgaagccgaagctgaccagcagatggacaacttgtacctgaaagccttggagggtttcattgccgtggtgacccaagatggcgacatgatctttctgtcagaaaacatcagcaagttcatgggacttacacaggtggagctaacaggacatagtatctttgacttcactcatccctgcgaccatgaggagattcgtgagaacctgagtctcaaaaatggctctggttttgggaaaaaaagcaaagacatgtccacagagcgggacttcttcatgaggatgaagtgcacggtcaccaacagaggccgtactgtcaacctcaagtcagccacctggaaggtcttgcactgcacgggccaggtgaaagtctacaacaactgccctcctcacaatagtctgtgtggctacaaggagcccctgctgtcctgcctcatcatcatgtgtgaaccaatccagcacccatcccacatggacatccccctggatagcaagaccttcctgagccgccacagcatggacatgaagttcacctactgtgatgacagaatcacagaactgattggttaccaccctgaggagctgcttggccgctcagcctatgaattctaccatgcgctagactccgagaacatgaccaagagtcaccagaacttgtgcaccaagggtcaggtagtaagtggccagtaccggatgctcgcaaagcatgggggctacgtgtggctggagacccaggggacggtcatctacaaccctcgcaacctgcagccccagtgcatcatgtgtgtcaactacgtcctgagtgagattgagaagaatgacgtggtgttctccatggaccagactgaatccctgttcaagccccacctgatggccatgaacagcatctttgatagcagtggcaagggggctgtgtctgagaagagtaacttcctattcaccaagctaaaggaggagcccgaggagctggcccagctggctcccaccccaggagacgccatcatctctctggatttcgggaatcagaacttcgaggagtcctcagcctatggcaaggccatcctgcccccgagccagccatgggccacggagttgaggagccacagcacccagagcgaggctgggagcctgcctgccttcaccgtgccccaggcagctgccccgggcagcaccacccccagtgccaccagcagcagcagcagctgctccacgcccaatagccctgaagactattacacatctttggataacgacctgaagattgaagtgattgagaagctcttcgccatggacacagaggccaaggaccaatgcagtacccagacggatttcaatgagctggacttggagacactggcaccctatatccccatggacggggaagacttccagctaagccccatctgccccgaggagcggctcttggcggagaacccacagtccaccccccagcactgcttcagtgccatgacaaacatcttccagccactggcccctgtagccccgcacagtcccttcctcctggacaagtttcagcagcagctggagagcaagaagacagagcccgagcaccggcccatgtcctccatcttctttgatgccggaagcaaagcatccctgccaccgtgctgtggccaggccagcacccctctctcttccatggggggcagatccaatacccagtggcccccagatccaccattacattttgggcccacaaagtgggccgtcggggatcagcgcacagagttcttgggagcagcgccgttggggccccctgtctctccaccccatgtctccaccttcaagacaaggtctgcaaagggttttggggctcgaggcccagacgtgctgagtccggccatggtagccctctccaacaagctgaagctgaagcgacagctggagtatgaagagcaagccttccaggacctgagcgggggggacccacctggtggcagcacctcacatttgatgtggaaacggatgaagaacctcaggggtgggagctgccctttgatgccggacaagccactgagcgcaaatgtacccaatgataagttcacccaaaaccccatgaggggcctgggccatcccctgagacatctgccgctgccacagcctccatctgccatcagtcccggggagaacagcaagagcaggttccccccacagtgctacgccacccagtaccaggactacagcctgtcgtcagcccacaaggtgtcaggcatggcaagccggctgctcgggccctcatttgagtcctacctgctgcccgaactgaccagatatgactgtgaggtgaacgtgcccgtgctgggaagctccacgctcctgcaaggaggggacctcctcagagccctggaccaggccacctga

SEQ ID NO: 36 - DNA, 1353 bp, Homo sapiens
atgggggaaccccgggctggggccgccctggacgatggcagcggctggacgggcagtgaggaaggcagtgaggagggtaccggcggcagtgagggggctgggggtgacgggggcccggatgcagagggggtgtggagcccagacattgagcagagcttccaggaggccctggccatctatccaccctgcggccgccggaaaataattttgtctgatgaaggcaagatgtatggtcggaatgaactgatcgcccgctacatcaagctgagaacggggaagacccgaactcgaaaacaggtttctagtcacatccaggttttggcccgaaggaaatcaagggaaatccagtccaagttgaaggaccaggtttccaaggacaaggctttccagacaatggcaaccatgtcctctgcccagctcatctccgcgccttctctgcaggccaaactgggtcccactggtcctcaggtggtccaggcctctgagcttttccagttttggtctggaggatctgggcccccctggaatgttccagatgtgaagccattctcacagacaccgttcaccttgtcactgactcccccatctactgacctcccagggtacgagcccccccaagccctctcacccctgcccccacctaccccatcgcccccagcctggcaggctcggggcctgggcaccgcccggttgcagctggtagagttctcagccttcgtggaaccgccagatgcagttgattcttaccagaggcacctgttcgtgcacatcagccagcactgccccagccccggagcgccgccgctcgagagtgtggacgtccggcagatctacgacaaattccctgagaaaaagggtggcctccgagagctatatgatcgtggccccccccatgccttcttcctggtcaagttctgggcggacctgaactggggcccaagtggtgaggaggcaggggccggtggcagcatcagcagtggtggcttctacggagtgagcagccagtatgagagcctggaacacatgaccctcacctgttcctccaaggtctgctcttttggcaagcaggtggtggagaaggtggagacggaacgggcccagctggaggacggcagatttgtgtaccgcctgctgcgctcgcccatgtgcgagtacctggtgaatttcttgcacaagttgcggcagctgcctgagcgatacatgatgaacagcgtcctggaaaacttcaccatcctccaggtggtgacaaacagagacacccaggaactgctgctctgcaccgcctatgtcttcgaggtctccaccagcgagcgtggggcccagcatcacatttaccgcctggtcagggactga

SEQ ID NO: 37 - DNA, 807 bp, Homo sapiens
atgccgcgctccttcctggtcaagaagcatttcaacgcctccaaaaagccaaactacagcgaactggacacacatacagtgattatttccccgtatctctatgagagttactccatgcctgtcataccacaaccagagatcctcagctcaggagcatacagccccatcactgtgtggactaccgctgctccattccacgcccagctacccaatggcctctctcctctttccggatactcctcatctttggggcgagtgagtccccctcctccatctgacacctcctccaaggaccacagtggctcagaaagccccattagtgatgaagaggaaagactacagtccaagctttcagacccccatgccattgaagctgaaaagtttcagtgcaatttatgcaataagacctattcaactttttctgggctggccaaacataagcagctgcactgcgatgcccagtctagaaaatctttcagctgtaaatactgtgacaaggaatatgtgagcctgggcgccctgaagatgcatattcggacccacacattaccttgtgtttgcaagatctgcggcaaggcgttttccagaccctggttgcttcaaggacacattagaactcacacgggggagaagcctttttcttgccctcactgcaacagagcatttgcagacaggtcaaatctgagggctcatctgcagacccattctgatgtaaagaaataccagtgcaaaaactgctccaaaaccttctccagaatgtctctcctgcacaaacatgaggaatctggctgctgtgtagcacactga

SEQ ID NO: 38 - DNA, 1281 bp, Homo sapiens
attgagcccagcagctggagcggcagtgagagccctgccgaaaacatggaaaggatgagtgactctgcagataagccaattgacaatgatgcagaaggggtctggagccccgacatcgagcaaagctttcaggaggccctggctatctatccaccatgtgggaggaggaaaatcatcttatcagacgaaggcaaaatgtatggtaggaatgaattgatagccagatacatcaaactcaggacaggcaagacgaggaccagaaaacaggtgtctagtcacattcaggttcttgccagaaggaaatctcgtgattttcattccaagctaaaggatcagactgcaaaggataaggccctgcagcacatggcggccatgtcctcagcccagatcgtctcggccactgccattcataacaagctggggctgcctgggattccacgcccgaccttcccaggggcgccggggttctggccgggaatgattcaaacagggcagccaggatcctcacaagacgtcaagccttttgtgcagcaggcctaccccatccagccagcggtcacagcccccattccagggtttgagcctgcatcggccccagctccctcagtccctgcctggcaaggtcgctccattggcacaaccaagcttcgcctggtggaattttcagcttttctcgagcagcagcgagacccagactcgtacaacaaacacctcttcgtgcacattgggcatgccaaccattcttacagtgacccattgcttgaatcagtggacattcgtcagatttatgacaaatttcctgaaaagaaaggtggcttaaaggaactgtttggaaagggccctcaaaatgccttcttcctcgtaaaattctgggctgatttaaactgcaatattcaagatgatgctggggctttttatggtgtaaccagtcagtacgagagttctgaaaatatgacagtcacctgttccaccaaagtttgctcctttgggaagcaagtagtagaaaaagtagagacggagtatgcaaggtttgagaatggccgatttgtataccgaataaaccgctccccaatgtgtgaatatatgatcaacttcatccacaagctcaaacacttaccagagaaatatatgatgaacagtgttttggaaaacttcacaattttattggtggtaacaaacagggatacacaagaaactctactctgcatggcctgtgtgtttgaagtttcaaatagtgaacacggagcacaacatcatatttacaggcttgtaaaggactga

(19) United States
(12) Patent Application Publication
(10) Pub. No.: US 2024/0165133 A1
(43) Pub. Date: May 23, 2024

(54) METHOD FOR SUPPRESSING CANCER METASTASIS THROUGH ALTERATION OF ADHESION DEPENDENCE OF CANCER CELLS

(71) Applicant: INDUSTRY-ACADEMIC COOPERATION FOUNDATION, YONSEI UNIVERSITY, Seoul (KR)

(72) Inventors: Hyun Woo PARK, Seoul (KR); Hyunbin HUH, Incheon (KR)

(73) Assignee: INDUSTRY-ACADEMIC COOPERATION FOUNDATION, YONSEI UNIVERSITY, Seoul (KR)

(21) Appl. No.: 18/003,968

(22) PCT Filed: Jul. 5, 2021
(86) PCT No.: PCT/KR2021/008524

§ 371 Date: Dec. 30, 2022

(30) Foreign Application Priority Data
Jul. 2, 2020 (KR) 10-2020-0081391

(51) Int. Cl. A61K 31/65; C12Q 1/6886; G01N 33/50

(52) U.S. Cl. CPC A61K 31/65; C12Q 1/6886; G01N 33/5011; C12Q 2600/136; C12Q 2600/158

(57) ABSTRACT

The present disclosure relates to a method for discovering factors determining adhesion dependence of cells and a method for preventing or treating cancer, specifically metastatic cancer by regulating the expression of the factors. The present disclosure suggests a completely new inhibitory target for cancer metastasis, which allows remarkable blocking of the production of circulating tumor cells from primary cancer tissues and, ultimately, provision of an effective anticancer composition capable of significantly reducing the mortality rate of cancer.

### TECHNICAL FIELD

The present disclosure relates to a method for inhibiting cancer metastasis by regulating the expression of factors changing the adhesion dependence of cells and thereby inhibiting the production of circulating tumor cells.

### BACKGROUND ART

Cancer metastasis refers to a phenomenon wherein cancer cells break away from primary tumor tissues and form new tumors at distant sites as they pass through blood or lymphatic vessels. Because 90% or more of the deaths of cancer patients are caused by metastasis from primary cancers (Nature Reviews Cancer, 2006, 6: 49-458), the inhibition of cancer metastasis is as important as the treatment of primary cancer in improving the mortality rate of cancer patients.

The mechanism whereby cancer cells acquire mobility during metastasis has not been elucidated completely yet and many theories have been suggested. The most actively studied theory is the EMT (epithelial to mesenchymal transition)/MET (mesenchymal to epithelial transition) theory, which states that tumor epithelial cells acquire the characteristics of mesenchymal cells due to genetic changes (J Clin Invest. 2009, 119: 1417-1419). The theory hypothesizes that epithelial cells that have acquired the characteristics of mesenchymal cells migrate from their original sites into blood vessels due to weakened intercellular adhesion and they recover their epithelial characteristics while circulating in the blood vessels (MET) and form tumors at distant secondary sites. However, many cases of cancer metastasis not accompanied by EMT are being reported recently. On the contrary, it is reported that E-cadherin, which is an inhibitory marker of EMT, facilitates cancer metastasis by increasing the survivability of circulating tumor cells. Therefore, it is unclear whether the inhibition of EMT can be an effective target for cancer metastasis.

Another theory explains metastasis with the presence of cancer stem cells. The theory states that cancer stem cells having stemness play key roles in tumor growth, metastasis, etc. in tumor tissues as in normal tissues. However, although the presence of cancer stem cells having distinguished characteristics from adult cancer cells have been identified in various researches, systemic metastasis by cancer stem cells has not been reproduced well in animal experiments (Int J Cancer. 2008; 123: 73-84).

Therefore, the inventors of the present disclosure have searched for specific genes that determine the phenotype of circulating tumor cells in order to present a new mechanism of cancer metastasis and propose a more effective target for inhibiting cancer metastasis. Unlike primary cancer cells that grow as being attached to extracellular matrices, the circulating tumor cells have anoikis resistance and are not adhesion-dependent. Therefore, they have investigated whether the gene expressed differently in adherent cells and suspension cells actually cause the metastasis of cancer cells.

Throughout the present specification, a number of papers and patent documents are referred to and their citations are indicated. The disclosed contents of the cited papers and patent documents are included herein by reference in their entirety, so that the related art and the contents of the present disclosure could be described more clearly.

### DISCLOSURE

### Technical Problem

The inventors of the present disclosure have made consistent efforts to search for effective inhibitory targets of cancer metastasis that cause death of cancer patients and develop a new therapeutic method capable of remarkably decreasing the mortality rate of cancer. As a result, they have identified core genes that determine the suspension phenotype of circulating tumor cells (CTCs) and found out that tumor metastasis can be inhibited effectively by artificially expressing or inhibiting the expression of genes differentiated exclusively depending on the adhesion dependence of cells, and have completed the present disclosure.

Accordingly, the present disclosure is directed to proving a composition for preventing or treating cancer.

The present disclosure is also directed to proving a composition for diagnosing the metastasis or recurrence of cancer.

The present disclosure is also directed to proving a method for screening a composition for preventing or treating cancer.

Other purposes and advantages of the present disclosure will become more apparent by the following detailed description, claims and drawings.

### Technical Solution

According to an aspect of the present disclosure, the present disclosure provides a composition for preventing or treating cancer, which contains an inhibitor of the expression of one or more gene selected from a group consisting of IKZF1, KLF1, IRF8, BTG2, SPIB, GATA1, IKZF3, TALI, EAF2, POU2F2, KLF2, SPI1, NFE2, AKNA, IRF5, TCF7, RHOXF2, MYB, BCL11A and GFI1B as an active ingredient.

The inventors of the present disclosure have made consistent efforts to search for effective inhibitory targets of cancer metastasis that cause death of cancer patients and develop a new therapeutic method capable of remarkably decreasing the mortality rate of cancer. As a result, they have identified core genes that determine the suspension phenotype of circulating tumor cells (CTCs) and found out that tumor metastasis can be inhibited effectively by artificially expressing or inhibiting the expression of genes differentiated exclusively depending on the adhesion dependence of cells, and have completed the present disclosure.

The inventors of the present disclosure have identified the genes that are expressed only in suspension cells and not expressed in adherent cells. They have identified that the cells in which the 20 genes described above are expressed artificially are transformed to suspension cells regardless of the original phenotype of the cells. The have also identified that, conversely, when the expression of the genes is inhibited, suspension cells are transformed to adherent cells. In addition, it was verified through experiments that the inhibition of the expression of the genes in cancer cells can lead to inhibition of CTC formation and tumor metastasis.

In the present specification, the term “expression inhibitor” refers to a substance which inhibits the activation or expression of a target gene, such that the detection of the activation or expression of the target gene is impossible or meaningless or the biological function of the target gene can be inhibited significantly.

The inhibitor of the target gene includes, for example, a shRNA, a siRNA, a miRNA, a ribozyme, a PNA (peptide nucleic acid) or an antisense oligonucleotide that inhibits the expression of the 20 factors in gene level, a CRISPR system including a guide RNA that recognizes the target genes, an antibody or an aptamer that inhibits the genes in protein level, and a compound, a peptide or a natural product that inhibits their activities. Any means for inhibiting in gene or protein level known in the art may be used without limitation.

In the present specification, the term “shRNA (small hairpin RNA)” refers to an RNA sequence consisting of 50-70 single-stranded nucleotides that form a stem-loop structure in vivo. It forms a tight hairpin structure for inhibiting the expression of a target gene through RNA interference. Usually, long RNAs of 19-29 nucleotides form base pairs complementarily on both sides of a loop of 5-10 nucleotides, thus forming a double-stranded stem. It is transduced into cells by a vector including a U6 promoter to ensure that it is expressed always. The vector is usually passed on to daughter cells, allowing the target gene silencing to be inherited.

In the present specification, the term “siRNA” refers to a short double-stranded RNA that can induce RNAi (RNA interference) by cleaving a specific mRNA. It consists of a sense RNA strand having a sequence corresponding to the mRNA of a target gene and an antisense RNA having a sequence complementary thereto. The overall length of the siRNA is 10-100 nucleotides, specifically 15-80 nucleotides, most specifically 20-70 nucleotides. The siRNA may have either a blunt or cohesive end as long as it enables the inhibition of the expression of the target gene via RNAi effect. The cohesive end may be prepared in 3′-end overhanging structure or 5′-end overhanging structure.

In the present specification, the term “miRNA (microRNA)” is an oligonucleotide not expressed in cells. It refers to a single-stranded RNA molecule which has a short stem-loop structure and inhibits the expression of a target gene through complementary binding to the mRNA of the target gene.

In the present specification, the term “ribozyme” refers to an RNA molecule having an activity of an enzyme which recognizes and cleaves a base sequence of a specific RNA. The ribozyme consists of a region binding specifically to a target mRNA strand and a region cleaving the target RNA.

In the present specification, the term “PNA (peptide nucleic acid)” refers to a molecule having the characteristics of both a nucleic acid and a protein, which is capable of binding complementarily to a DNA or a RNA. The PNA is not found in nature but is synthesized artificially by a chemical method. The PNA regulates the expression of a target gene by forming a double strand through hybridization with a natural nucleic acid having a complementary base sequence.

In the present specification, the term “antisense oligonucleotide” refers to a nucleic acid molecule which binds to the complementary sequence of a target mRNA as a nucleotide complementary to the mRNA and inhibits essential activities for translation into a protein, translocation into the cytoplasm, maturation or other biological functions.

The antisense oligonucleotide may be modified at one or more base, sugar or backbone for higher inhibition efficiency (De Mesmaeker et al., Curr Opin Struct Biol., 5(3): 343-55, 1995). The oligonucleotide backbone may be modified with phosphorothioate, phosphotriester, methyl phosphonate, short-chain alkyl, cycloalkyl, single-chain heteroatomic or heterocyclic bond between sugar moieties.

According to the present disclosure, the expression inhibitor of the present disclosure may be a specific antibody that inhibits the activity of the proteins encoded by the genes. The antibody that specifically recognizes the target protein may be a polyclonal or monoclonal antibody, specifically a monoclonal antibody.

The antibody of the present disclosure may be prepared by methods commonly used in the art, for example, fusion method (Kohler and Milstein, European Journal of Immunology, 6: 511-519 (1976)), recombinant DNA method (U.S. Pat. No. 4,816,567) or phage antibody library method (Clackson et al, Nature, 352: 624-628 (1991) and Marks et al, J. Mol. Biol., 222: 58, 1-597 (1991)). General procedures for antibody production are described in detail in Harlow, E. and Lane, D., Using Antibodies: A Laboratory Manual, Cold Spring Harbor Press, New York, 1999; and Zola, H., Monoclonal Antibodies: A Manual of Techniques, CRC Press, Inc., Boca Raton, Florida, 1984.

In the present disclosure, an aptamer binding specifically to a target protein may be used instead of the antibody to inhibit the activity of the target protein. In the present specification, the term “aptamer” refers to a single-stranded nucleic acid (RNA or DNA) molecule or a peptide molecule binding specifically to a specific target substance with high affinity. Generals about the aptamer are described in detail in Hoppe-Seyler F, Butz K “Peptide aptamers: powerful new tools for molecular medicine”. J Mol Med. 78(8): 426-(2000); and Cohen BA, Colas P, Brent R. “An artificial cell-cycle inhibitor isolated from a combinatorial library”. Proc Natl Acad Sci USA. 95(24): 14272-7 (1998).

In the present specification, the term “prevention” refers to prevention of the onset of a disorder or a disease in a subject who has not been diagnosed with the disorder or disease but has the risk of contracting the disorder or disease.

In the present specification, the term “treatment” refers to (a) prevention of the development of a disorder, a disease or symptoms; (b) alleviation of a disorder, a disease or symptoms; or (c) removal of a disorder, a disease or symptoms. When the composition of the present disclosure is administered to a subject, the development of symptoms owing to tumor, specifically metastatic tumor, is prevented, removed or alleviated as the expression of the 20 genes described above or the proteins encoded by them is inhibited and the production of circulating tumor cells is inhibited. Accordingly, the composition of the present disclosure may be used as a composition for treating a disease or may be used as a therapeutic adjuvant together with another pharmacological ingredient. Thus, in the present specification, the term “treatment” or “therapeutic agent” includes the meaning of “assistance of treatment” or “therapeutic adjuvant”.

In the present specification, the term “administration” or “administer” refers to direct administration of a therapeutically effective amount of the composition of the present disclosure to a subject so that the same amount is formed in the body of the subject.

In the present disclosure, the term “therapeutically effective amount” refers to the amount of the pharmaceutical composition of the present disclosure which is enough for the pharmacological ingredient in the composition to provide a therapeutic or prophylactic effect in a subject and, thus, includes “prophylactically effective amount”.

In the present specification, the term “subject” includes human, mouse, rat, guinea pig, dog, cat, horse, cow, pig, monkey, chimpanzee, baboon or rhesus monkey without limitation. Specifically, the subject of the present disclosure is human.

According to a specific exemplary embodiment of the present disclosure, the composition of the present disclosure contains an expression inhibitor of the BTG2 and IKZF1 genes.

According to a more specific exemplary embodiment of the present disclosure, the composition of the present disclosure further contains an expression inhibitor of the NFE2, IRF8 and SPIB genes. Most specifically, it further contains an expression inhibitor of the GATA1, IKZF3, TALI, EAF2 and POU2F2 genes.

According to another aspect of the present disclosure, the present disclosure provides a composition for preventing or treating cancer, which contains a nucleotide of one or more gene selected from a group consisting of TSC22D1, VAX2, SOX13, ARNT2, PPARG, BNC2, HOXD8, GLIS3, FOXD8, RARG, MEIS3, TGFB111, TBX3, SOX9, EPAS1, TEAD2, SNA12 and TEAD1 as an active ingredient.

In the present specification, the term “nucleotide” includes DNA (gDNA and cDNA) and RNA molecules comprehensively. The nucleotide, which is a monomeric unit of a nucleic acid molecule, includes not only natural nucleotides but also analogues with modified sugar or base moieties. In the present disclosure, it is obvious to those skilled in the art that the nucleotide sequence whose the expression level is to be measured is not limited to those described in the attached sequence listings. Some of the modifications of the nucleotide lead to change in proteins. The nucleotide encompasses all nucleotide molecules having functionally equivalent codons, codons encoding the same amino acids due to codon degeneracy, or codons encoding biologically equivalent amino acids.

When considering biologically equivalent variations described above, the nucleotide whose the expression level is to be measured in the present disclosure is understood to encompass the sequences having substantial identity to the genes described above. The sequences having substantial identity mean the sequences that show at least 70% of identity, specifically 80% of identity, more specifically 90% of identity, most specifically 95% of identity, to the sequences of the genes when aligned to match as much as possible and analyzed using an algorithm commonly used in the art. Methods for aligning sequences for comparison are well known in the art. Various methods and algorithms for the alignment are described in Huang et al., Comp. Appl. BioSci. 8: 155-65 (1992) and Pearson et al., Meth. Mol. Biol. 24: 307-31 (1994). NCBI Basic Local Alignment Search Tool (BLAST) (Altschul et al., J. Mol. Biol. 215: 403-10 (1990)) is available from several sources, including the NBCI (National Center for Biological Information) and the Internet, for use in connection with sequence analysis programs such as blastp, blasm, blastx, tblastn and tblastx.

According to a specific exemplary embodiment of the present disclosure, the cancer that can be prevented or treated with the composition of the present disclosure is cancer before production of circulating tumor cells (CTCs), and the composition of the present disclosure inhibits the production of circulating tumor cells.

According to the present disclosure, if the expression of the 20 genes that confer suspension phenotype to cells is inhibited or if 18 genes that confer adherent phenotype is overexpressed, the production of circulating tumor cells is blocked by preventing the cells from adapting to the suspension environment. Through this, the metastasis or recurrence of cancer mediated by circulating tumor cells can be inhibited effectively. Accordingly, the composition of the present disclosure may also be expressed as a “composition for preventing or treating cancer metastasis” or a “composition for inhibiting cancer metastasis”.

According to another aspect of the present disclosure, the present disclosure provides a composition for preventing or treating cancer wherein circulating tumor cells (CTCs) have been produced, which contains a nucleotide of one or more gene selected from a group consisting of IKZF1, KLF1, IRF8, BTG2, SPIB, GATA1, IKZF3, TALI, EAF2, POU2F2, KLF2, SPI1, NFE2, AKNA, IRF5, TCF7, RHOXF2, MYB, BCL11A and GFI1B as an active ingredient.

According to another aspect of the present disclosure, the present disclosure provides a composition for preventing or treating cancer wherein circulating tumor cells (CTCs) have been produced, which contains an inhibitor of the expression of one or more gene selected from a group consisting of TSC22D1, VAX2, SOX13, ARNT2, PPARG, BNC2, HOXD8, GLIS3, FOXD8, RARG, MEIS3, TGFB111, TBX3, SOX9, EPAS1, TEAD2, SNAI2 and TEAD1 as an active ingredient.

Tumor metastasis is achieved as follows. Cancer cells detached from primary cancer tissues migrate into blood vessels and then circulate through the bloodstream to new sites, forming secondary metastatic cancer as they are attached to the tissues of the sites, are divided and grow. The inventors of the present disclosure have found out that, although it is effective to inhibit the production of circulating tumor cells to interrupt this process called distant metastasis, if circulating tumor cells have already been produced and have invaded into blood vessels, it is more effective to artificially maintain the suspension phenotype to prevent the attachment of the cells to the secondary sites. Accordingly, if the presence of CTCs in vivo has been detected, the colonization of the produced circulating tumor cells can be prevented and, ultimately, the formation of metastatic cancer can be interrupted effectively by overexpressing the 20 genes that confer suspension phenotype to cells or inhibiting the 18 genes that confer adherent phenotype.

According to another aspect of the present disclosure, the present disclosure provides a composition for diagnosing the metastasis or recurrence of cancer, which contains an agent for measuring the expression of one or more gene selected from a group consisting of IKZF1, KLF1, IRF8, BTG2, SPIB, GATA1, IKZF3, TALI, EAF2, POU2F2, KLF2, SPI1, NFE2, AKNA, IRF5, TCF7, RHOXF2, MYB, BCL11A, GFI1B, TSC22D1, VAX2, SOX13, ARNT2, PPARG, BNC2, HOXD8, GLIS3, FOXD8, RARG, MEIS3, TGFB111, TBX3, SOX9, EPAS1, TEAD2, SNAI2 and TEAD1 as an active ingredient.

The agent for measuring the expression of the gene may be a primer or a probe binding specifically to a nucleic acid molecule of the gene.

In the present specification, the term “nucleic acid molecule” includes DNA (gDNA and cDNA) and RNA molecules comprehensively and the nucleotide, which is a monomeric unit of a nucleic acid molecule, includes not only natural nucleotides but also analogues with modified sugar or base moieties (Scheit, Nucleotide Analogs, John Wiley, New York (1980); Uhlman and Peyman, Chemical Reviews, 90: 543-584 (1990)).

The term “primer” used in the present specification refers to an oligonucleotide which acts as a point of initiation of synthesis when placed under conditions in which the synthesis of a primer extension product which is complementary to a nucleic acid strand (template) is induced, i.e., in the presence of nucleotides and an agent for polymerization such as DNA polymerase and at suitable temperature and pH. Specifically, the primer is a single-stranded deoxyribonucleotide. The primer used in the present disclosure may include naturally occurring dNMPs (i.e., dAMP, dGMP, dCMP and dTMP), modified nucleotides, non-naturally occurring nucleotides, and ribonucleotides.

The primer of the present disclosure may be an extension primer that is annealed to a target nucleic acid to form a sequence complementary to the target nucleic acid by a template-dependent nucleic acid polymerase. The extension primer is extended to a site at which an immobilized probe is annealed and, thus, occupies the site at which the probe is annealed.

The extension primer used in the present disclosure includes a hybrid nucleotide sequence complementary to a specific base sequence of a target nucleic acid, e.g., the genes described above. The term “complementary” refers to being sufficiently complementary such that a primer or a probe can be selectively hybridized with the target nucleic acid sequence under a predetermined annealing or hybridization condition, and encompasses substantially complementary and perfectly complementary. Specifically, it means perfectly complementary. In the present specification, the term “substantially complementary sequence” includes not only a perfectly matched sequence but also a sequence that is partially unmatched to a compared sequence, within a range in which the sequence can be annealed to the sequence to serve as a primer.

The primer should be long enough to prime the synthesis of an extension product in the presence of a polymerase. The appropriate length of the primer varies depending on several factors, such as temperature, pH and primer source, but the primer is typically 15-30 nucleotides in length. Short primer molecules generally require lower temperatures in order to form sufficiently stable hybrid complexes with templates. The design of the primer may be easily carried out by those skilled in the art with reference to the target nucleotide sequence. For instance, the primer may be designed using a program for primer design (e.g., PRIMER 3).

In the present specification, the term “probe” refers to a linear oligomer having a natural or modified monomer or linkage that can be hybridized with a specific nucleotide sequence, including a deoxyribonucleotide and a ribonucleotide. Specifically, the probe is single-stranded for maximum efficiency of hybridization. More specifically, it is a deoxyribonucleotide. The probe used in the present disclosure may be a sequence perfectly complementary to the specific base sequence of the genes described above, but a substantially complementary sequence may also be used as long as specific hybridization is not interrupted. In general, it is preferred to use a probe complementary to the 3′-end or 5′-end of the target sequence since the stability of a duplex formed by hybridization tends to be determined by the matching of terminal sequences.

Appropriate conditions for hybridization can be determined by referring to Joseph Sambrook, et al., Molecular Cloning, A Laboratory Manual, Cold Spring Harbor Laboratory Press, N.Y. (2001) and Haymes, B. D., et al., Nucleic Acid Hybridization, A Practical Approach, IRL Press, Washington, D.C. (1985).

The agent for measuring the expression of the gene may be an antibody or an aptamer which binds specifically to proteins encoded by the genes described above and measures their expression in protein level.

According to the present disclosure, the proteins encoded by the genes of the present disclosure may be detected by immunoassay using antigen-antibody reactions for analysis of the risk of cancer metastasis or recurrence in individuals. The immunoassay may be conducted according to previously developed various immunoassay or immunostaining protocols.

For example, when radioimmunoassay is conducted in the present disclosure, antibodies labeled with radioisotopes (e.g., C¹⁴, I¹²⁵, P³² and S³⁵) may be used. The risk of cancer metastasis or recurrence may be predicted by analyzing the intensity of final signals during the immunoassay.

In the present disclosure, an aptamer binding specifically to a target protein may be used instead of an antibody. Details about the aptamer will be omitted to avoid redundancy.

In the present specification, the term “diagnosis” encompasses the decision whether cancer metastasis or recurrence has occurred in an individual and the decision of a prognosis related with the future risk of cancer metastasis or recurrence in an individual in which cancer metastasis or recurrence has not occurred yet. Accordingly, the term “diagnosis of cancer metastasis or recurrence” may also be expressed as “prediction of the risk of cancer metastasis or recurrence”.

According to a specific exemplary embodiment of the present disclosure, if the expression of one or more gene selected from a group consisting of IKZF1, KLF1, IRF8, BTG2, SPIB, GATA1, IKZF3, TALI, EAF2, POU2F2, KLF2, SPI1, NFE2, AKNA, IRF5, TCF7, RHOXF2, MYB, BCL11A and GFI1 B has increased, it may be determined that the risk of cancer metastasis or recurrence has increased due to production of circulating tumor cells (CTCs).

According to a specific exemplary embodiment of the present disclosure, if the expression of one or more gene selected from a group consisting of TSC22D1, VAX2, SOX13, ARNT2, PPARG, BNC2, HOXD8, GLIS3, FOXD8, RARG, MEIS3, TGFB1I1, TBX3, SOX9, EPAS1, TEAD2, SNAI2 and TEAD1 has decreased, it may be determined that the risk of cancer metastasis or recurrence has increased due to production of circulating tumor cells (CTCs).

In the present disclosure, the term “increased expression” used while mentioning the “composition for diagnosing the metastasis or recurrence of cancer” means significantly increased expression of the corresponding gene as compared to a control group or a normal group, specifically increased expression by about 10% or more, about 20% or more, about 30% or more, about 40% or more, about 50% or more or about 60% or more as compared to the control group or the normal group, although not being limited thereto. In addition, the term “decreased expression” means significantly decreased expression of the corresponding gene as compared to a control group or a normal group, specifically decreased expression by about 10% or more, about 20% or more, about 30% or more, about 40% or more, about 50% or more or about 60% or more as compared to the control group or the normal group, although not being limited thereto.

According to a specific exemplary embodiment of the present disclosure, the composition of the present disclosure contains agents for measuring the expression of the BTG2 and IKZF genes.

According to a more specific exemplary embodiment of the present disclosure, the composition of the present disclosure further contains agents for measuring the expression of the NFE2, IRF8 and SPIB genes. Most specifically, it further contains agents for measuring the expression of the GATA1, IKZF3, TALI, EAF2 and POU2F2 genes.

According to another aspect of the present disclosure, the present disclosure provides a method for screening an inhibitor of the production of circulating tumor cells (CTCs) or an inhibitor of the colonization of produced circulating tumor cells, which includes:
(a) a step of contacting a test substance with a biological sample containing one or more gene selected from a group consisting of IKZF1, KLF1, IRF8, BTG2, SPIB, GATA1, IKZF3, TALI, EAF2, POU2F2, KLF2, SPI1, NFE2, AKNA, IRF5, TCF7, RHOXF2, MYB, BCL11A, GFI1B, TSC22D1, VAX2, SOX13, ARNT2, PPARG, BNC2, HOXD8, GLIS3, FOXD8, RARG, MEIS3, TGFB111, TBX3, SOX9, EPAS1, TEAD2, SNAI2 and TEAD1 or a protein encoded thereby; and
(b) a step of measuring the expression or activation of the gene or the protein in the sample, wherein
if the expression or activation of one or more gene selected from a group consisting of IKZF1, KLF1, IRF8, BTG2, SPIB, GATA1, IKZF3, TALI, EAF2, POU2F2, KLF2, SPI1, NFE2, AKNA, IRF5, TCF7, RHOXF2, MYB, BCL11A and GFI1B or a protein encoded thereby has decreased or if the expression or activation of one or more gene selected from a group consisting of TSC22D1, VAX2, SOX13, ARNT2, PPARG, BNC2, HOXD8, GLIS3, FOXD8, RARG, MEIS3, TGFB111, TBX3, SOX9, EPAS1, TEAD2, SNAI2 and TEAD1 or a protein encoded thereby has increased, the test substance is determined as an inhibitor of the production of circulating tumor cells (CTCs), and
if the expression or activation of one or more gene selected from a group consisting of IKZF1, KLF1, IRF8, BTG2, SPIB, GATA1, IKZF3, TALI, EAF2, POU2F2, KLF2, SPI1, NFE2, AKNA, IRF5, TCF7, RHOXF2, MYB, BCL11A and GFI1B or a protein encoded thereby has increased or if the expression or activation of one or more gene selected from a group consisting of TSC22D1, VAX2, SOX13, ARNT2, PPARG, BNC2, HOXD8, GLIS3, FOXD8, RARG, MEIS3, TGFB111, TBX3, SOX9, EPAS1, TEAD2, SNAI2 and TEAD1 or a protein encoded thereby has increased, the test substance is determined as an inhibitor of the colonization of produced circulating tumor cells.

According to a specific exemplary embodiment of the present disclosure, the biological sample contains cancer cells.

According to a specific exemplary embodiment of the present disclosure, the cancer is monastic or recurrent cancer.

Details about the adhesion dependence-regulating factors and the cancers that can be prevented or treated by regulating their expressions will be omitted to avoid redundancy.

In the present disclosure, the term “biological sample” refers to a sample containing cells that express the genes described above, which is obtained from a mammal including human, and includes a tissue, an organ, a cell or a cell culture, although not being limited thereto. More specifically, the biological sample may be a cancer tissue, a cancer cell or a culture thereof.

The term “test substance” used while mentioning the screening method of the present disclosure means an unknown substance used for screening, which is added to a sample containing cells that express the genes of the present disclosure in order to investigate whether it affects the activation or expression of those genes. The test substance includes a compound, a nucleotide, a peptide and a natural extract, although not being limited thereto. The step of measuring the expression or activation of the genes in the biological sample treated with the test substance may be performed by various methods for measuring expression or activation known the art.

### Advantageous Effects

The features and advantages of the present disclosure may be summarized as follows:
(a) The present disclosure provides a method for discovering factors determining adhesion dependence of cells and a method for preventing or treating cancer, specifically metastatic cancer by regulating the expression of the factors.
(b) The present disclosure suggests a completely new inhibitory target for cancer metastasis, which allows remarkable blocking of the production of circulating tumor cells from primary cancer tissues and, ultimately, provision of an effective anticancer composition capable of significantly reducing the mortality rate of cancer.

### BRIEF DESCRIPTION OF DRAWINGS

FIGS. 1a-1g show a procedure of selecting genes expressed mutually exclusively in adherent cells and suspension cells from the ENCODE database as candidates for AST and SAT. FIG. 1 a schematically summarizes the strategy for analysis of the ENCODE database for 131 adherent and suspension cells. FIG. 1b shows the volcano plot of genes expressed at high or low levels in suspension cells. FIG. 1c shows the cleavage map of the genes selected from the red dots in the volcano plot of FIG. 1b. FIG. 1d shows a result of conducting association analysis for the 1491 genes of the 112 adherent cells and the 21 suspension cells of the volcano plot of FIG. 1b. FIG. 1e schematically summarizes the strategy for selection of 20 ASTs and 18 SATs from the ENCODE and Proteinatlas.org databases. FIG. 1f shows a cleavage map for the expression of the 20 AST and 18 SAT candidate factors in the 112 adherent cells and the 21 suspension cells. FIG. 1g shows the cleavage map for the average of the 20 AST and 18 SAT candidate factors.

FIGS. 2a-2l show that the identified AST factors reprogram adhesion dependence. FIG. 2a schematically summarizes the strategy of inducing AST-SAT through lentiviral infection. FIG. 2b shows the morphologies of HEK293A cells that stably express mock or 20 AST factors. FIG. 2c shows an immunoblotting assay result for 20 AST candidate factors in HEK293A cells. FIG. 2d shows a LIVE/DEAD assay result for mock and 20 AST-HEK293A cells treated with puromycin (4 mg/mL). FIG. 2e shows the growth curves of mock and AST-reprogrammed HEK293A cells. FIG. 2f shows the Venn diagram for AST candidate factors expressed in AST-induced cells. FIG. 2g shows the morphology of HEK293A cells that stably express mock or 10 AST factors. FIG. 2h shows the effect of the removal of each of 20 AST factors on the production of AST-induced HEK293A cells. FIG. 2i shows the morphology of HEK293A cells that stably express mock or 5 AST factors. FIG. 2j shows the effect of the removal of each of 20 AST factors on the production of AST-induced HEK293A cells. FIG. 2k shows the volcano plot of genes expressed at high or low levels in suspension cells and the location of 5 AST factors. FIG. 2l shows the morphology of SUIT2, MDA-MB-231 and HEK293T cells that express mock and 5 AST factors. Data are representative values of three independent experiments.

FIGS. 3a-3d illustrate ‘in vitro CTC assay’ for predicting the spread of tumor cells and production of CTCs. FIG. 3a schematically shows an ‘in vitro CTC assay’ model of representing production and metastasis of CTCs. FIG. 3b shows the morphology of various cell lines in colony assay, suspension assay (methyl cellulose assay) and in vitro CTC assay. FIG. 3c shows the images of live MCF-7, HS578T, MDA-MB-231 and SUIT-2 cells treated with Pl. FIG. 3d shows the morphology of MDA-MB-231, SUIT-2 and AGS cells during the production of CTCs in vitro.

FIGS. 4a-4i show that the transient expression of AST factors in circulating tumor cells is essential for the spread of the tumor cells. FIG. 4a shows a qRT-PCR analysis result for several AST factors in parent cells (P), in vitro circulating tumor cells (CTC) and secondary cells (S) of MDA-MB-231 cells. FIG. 4b shows a qRT-PCR analysis result for several AST factors in parent cells (P), in vitro circulating tumor cells (CTC) and secondary cells (S) of SUIT-2 cells. FIG. 4c shows a qRT-PCR analysis result for several AST factors in parent cells (P), in vitro circulating tumor cells (CTC) and secondary cells (S) of AGS cells. FIGS. 4d and 4e show the effect of the introduction of nM siRNA on the production of in vitro CTCs in MDA-MB-231 and SUIT2 cells. FIGS. 4f and 4g show MCF10A and SUIT2 cells that stably express mock or 5 AST #1 factors. The SUIT2 cells were treated with 1 mM valproic acid. FIG. 4h shows AGS cells that stably express mock or 4 AST factors. FIG. 4i shows a qRT-PCR analysis result for EMT factors (CDH1 and CDH2) in parent cells (P), in vitro circulating tumor cells (CTC) and secondary cells (S) of SUIT-2 and AGS cells. FIG. 4i shows MDA-MB-231 cells that stably express mock or 4 AST factors.

FIGS. 5a-5b show that, after circulating tumor cells have been produced, tumor metastasis is inhibited when the AST factors of the present disclosure are activated. FIG. 5a shows the morphology of HEK293A cells that stably express TetR and 20 AST candidate factors when treated with doxycycline (5 mg/mL). FIG. 5b shows an immunoblotting result for 20 AST candidate factors in TetR-expressing HEK293A cells when treated with doxycycline.

[Best Mode]

Hereinafter, present disclosure is described in more detail through examples. The examples are provided only to describe the present disclosure more specifically and it will be obvious to those having ordinary knowledge in the art that the scope of the present disclosure is not limited by the examples.

### EXAMPLES

Experimental Methods

DNA Construct

Candidate human AST genes were tagged with V5 and FLAG and then subcloned into a pENTR4 vector (Addgene) which is a gateway insertion vector. A lentiviral expression vector was constructed by recombining the subcloned pENTR4 vector with a pLentiCMV vector which is a target vector using an LR recombinase (Invitrogen, 1179019). The structure of the construct was identified by sequencing.

Cell Culturing

All cells were maintained in a humidified 5% CO₂ incubator at 37° C. HEK293A, HEK293T, MCF7, MDA-MB-231, HS578T, HT-29, SW620, HCT116 and A375 cells were cultured in DMEM (Hyclone, SH30243), and BT549, SUIT-2, ASPC-1, MiaPaCa, AGS and MKN28 cells were cultured in RPMI (Hyclone, SH) containing 10% FBS (Hyclone, 1) and 50 μg/mL penicillin/streptomycin (Invitrogen, 15140122). MCF10A cells were cultured in DMEM-F12 supplemented with 5% horse serum (Invitrogen, 26050088), 20 ng/mL EGF (Peprotech, AF-100-15), 0.5 μg/mL hydrocortisone (Sigma, H4001-25G), 100 ng/mL cholera toxin (Sigma, C8052-2MG) and 10 μg/mL insulin (Sigma, 11882-100MG). Any of the cells of the present disclosure was found in the ICLAC and NCBI BioSample databases. It was confirmed that the cells were not contaminated by mycoplasma.

Viral Infection

HEK 293T cells were transfected with a lentiviral vector in which pMD2G- and psPAX2-encoding plasmid and construct were cloned using a Polyplus reagent (Merck) according to the manufacturer's instructions. 48 hours after the transfection, the medium containing virus particles was filtered with a 0.45-μm filter and then 8 μg/mL Polybrene was added. 24 hours later, the transfected cells were cultured in a fresh medium for 24 hours and then screened with puromycin and blasticidin.

Induction of Adherent-to-Suspension Transition (AST)

After seeding HEK293A cells (5×10⁵ cells) on a 6-well culture plate, the medium containing virus particles encoding the candidate AST genes was added. 2 days later, the transfected cells were trypsinized, seeded onto a new plate and then screened by treating with puromycin (4 mg/mL).

Antibodies

For western blot analysis, the following antibodies were used after dilution: anti-FLAG (Sigma-Aldrich), anti-V5 (Cell Signaling), anti-E-cadherin (Abcam), anti-N-cadherin (Abcam), anti-vimentin (Cell Signaling), anti-actin, anti-IKZF1, anti-BTG2, anti-IRF8 anti-NFE2 and anti-TALI.

Quantitative Real-Time PCR Analysis

RNA was extracted using an RNeasy Plus mini kit (QIAGEN, 74136). cDNA was obtained by conducing reverse transcription of the RNA sample using an iScript reverse transcriptase (Bio-Rad, 1708891). qRT-PCR was conducted using a KAPA SYBR FAST qPCR kit (Kapa Biosystems, KK4605) and a 7300 real-time PCR system (Applied Biosystems).

Statistical Analysis

All experiments were repeated at least 3 times and data were represented as mean±standard deviation. The statistical difference between two means was tested by two-sided unpaired Student's t-test. P<0.05 was regarded as statistically significant. No sample was excluded from analysis. The data showed a normal distribution and the compared groups had similar variances. Sample size was determined based on the results of previous studies.

Experimental Results

Selection of AST and SAT Candidate Factors from ENCODE Database

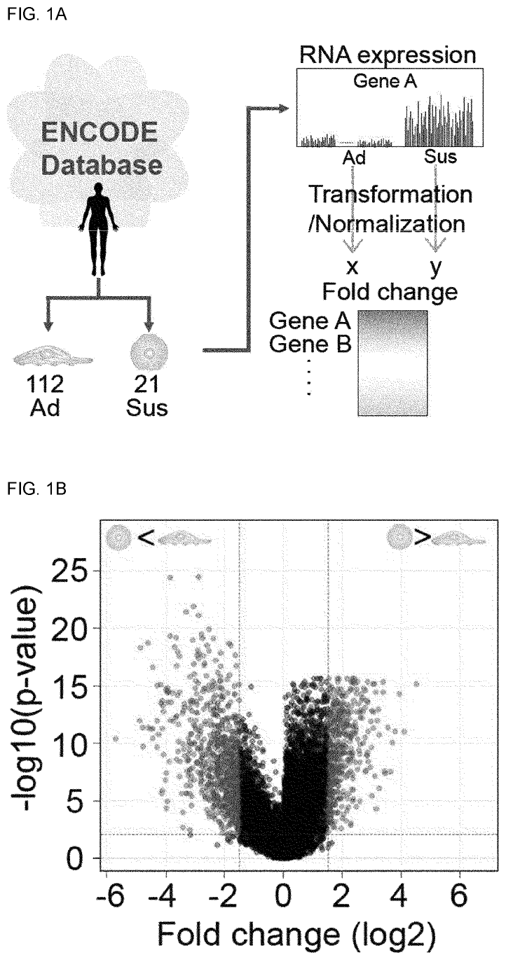

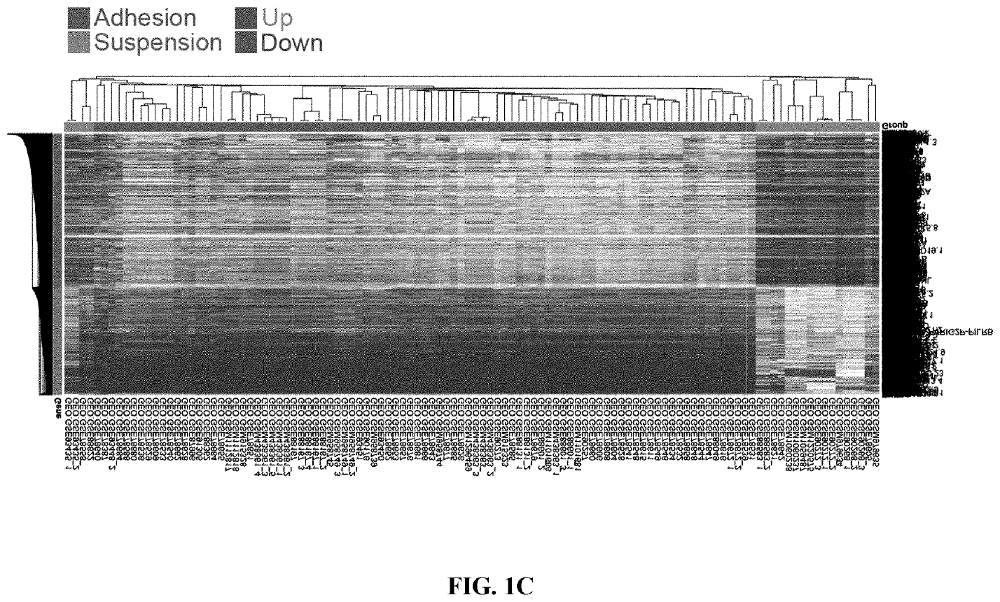

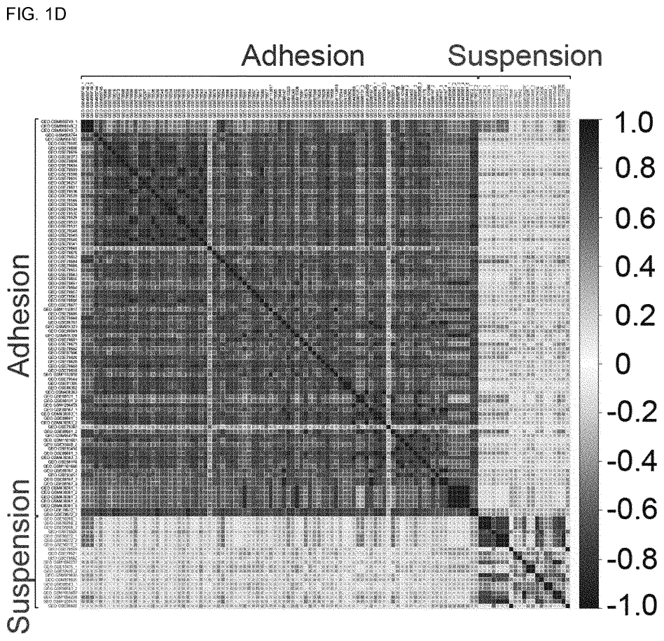

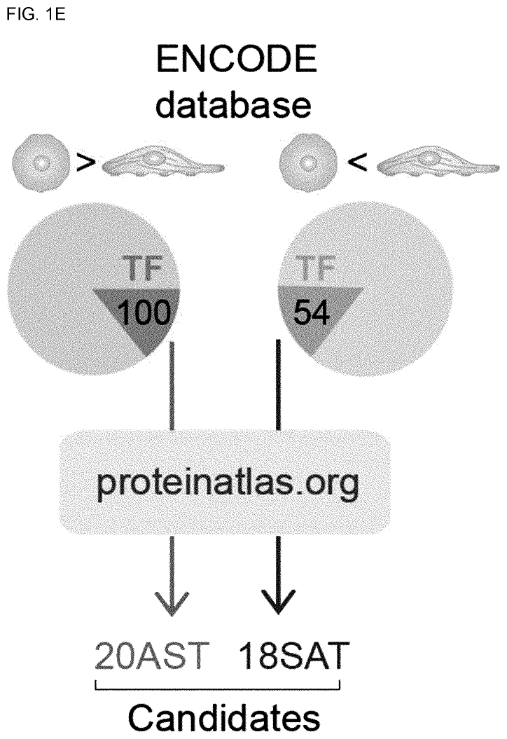

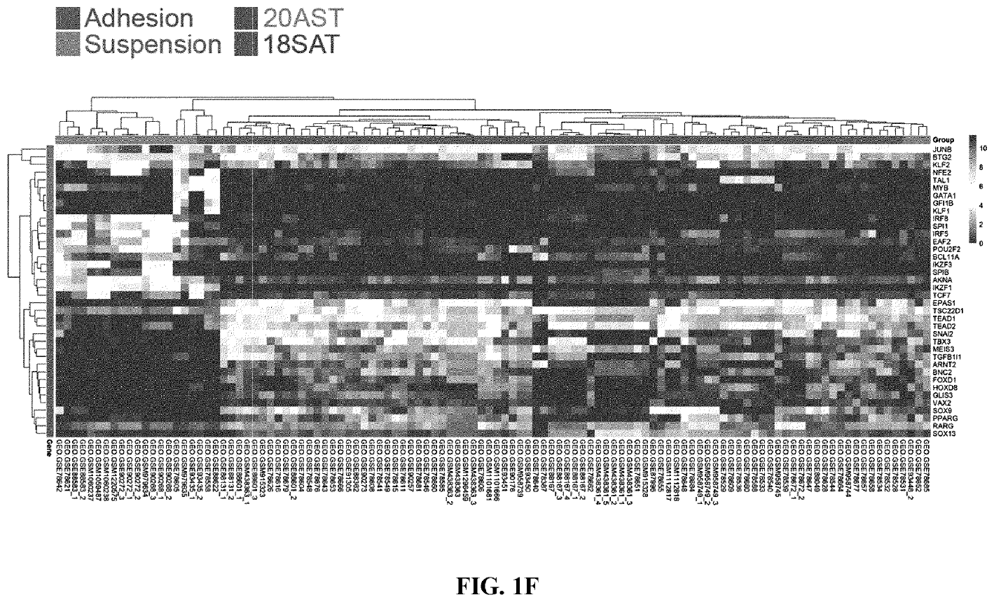

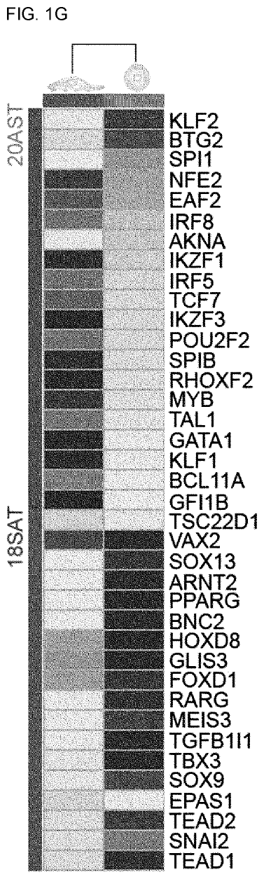

In order to screen the genes that are expressed mutually exclusively in adherent cells and suspension cells, 112 adherent cells and 21 suspension cells were selected from the ENCODE database. The RNA expression pattern of all the genes of suspension cells was compared with adherent cells (FIG. 1a). Particularly, as a result of RNA-seq screening, it was confirmed from the volcano plot that 654 and 862 genes were expressed at remarkably high level in adherent and suspension cells, respectively (FIG. 1b). As a result of visualizing the expression of the genes that show significant differences based on the volcano plot using a cleavage map, the clustering pattern of the cells depending on adhesion dependence could be identified (FIG. 1c). In addition, the genes selected by showing difference expression patterns in the adherent cells and the suspension cell were correlated with a Pearson correlation coefficient>0.1 (FIG. 1d). The linear correlation between the cells in the adhesion network was inferred using these genes. It was predicted that the adhesion of the cells to the ECM (extracellular matrix) would be determined by several transcription factors. In order to test this hypothesis, 20 and 19 genes that encode transcription factors and show mutually exclusive expression patterns in suspension cells or adherent cells were selected as candidate factors for adherent-to-suspension transition (AST) or suspension-to-adherent transition (SAT) from the Proteinatlas.org. database, respectively (FIG. 1e). Interestingly, in the cleavage maps, the expression pattern of the AST or SAT gene was biased mainly toward suspension cells or adherent cells (FIGS. 1f and 1g).

Reprogramming of Adhesion Dependence Through Specification of AST Factors

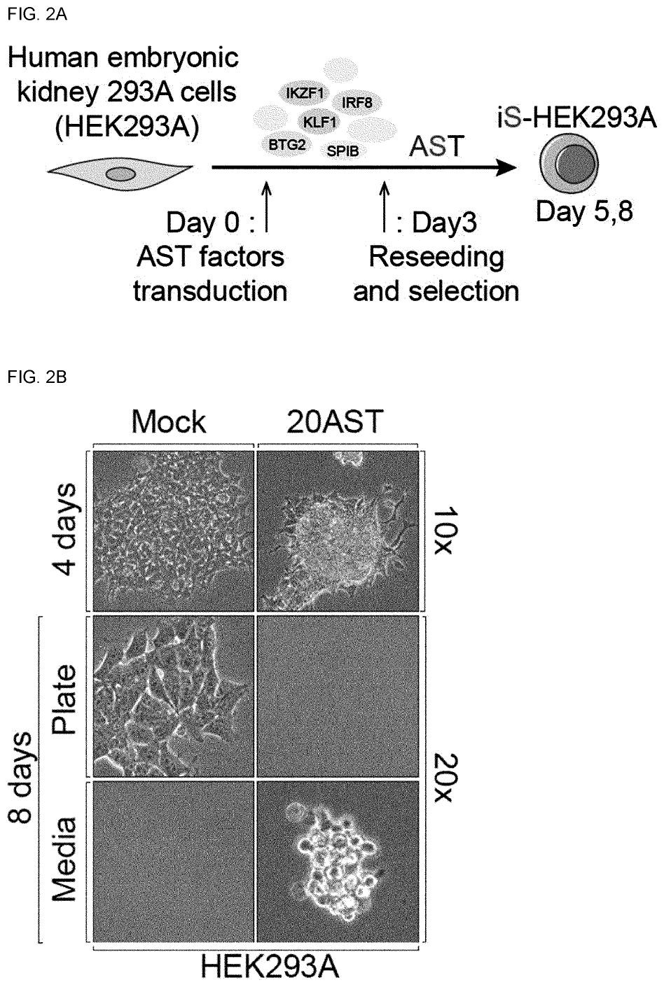

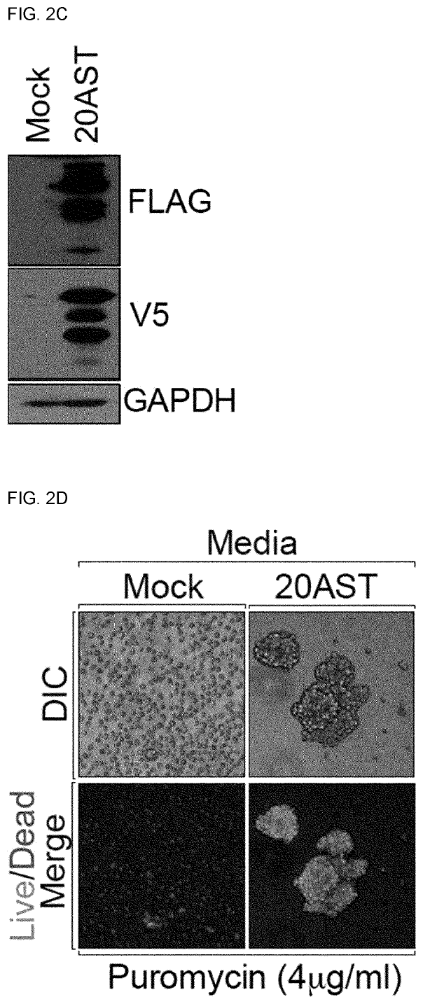

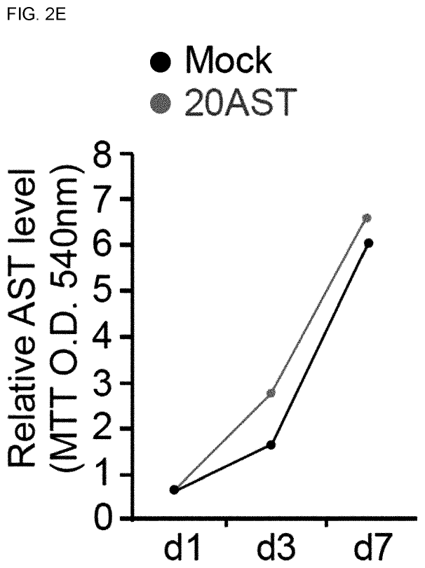

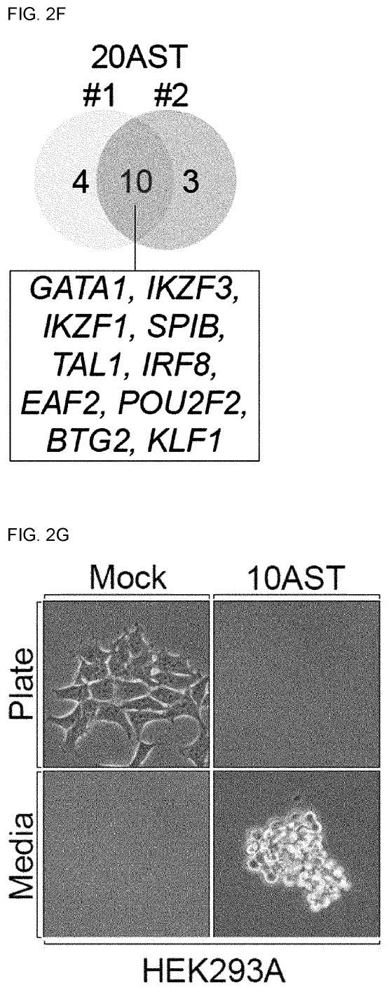

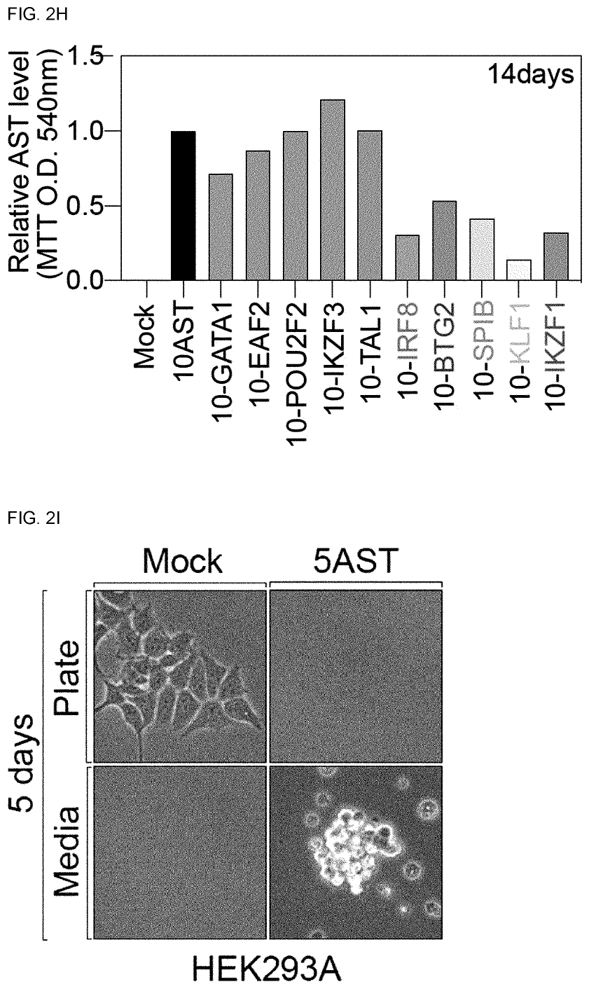

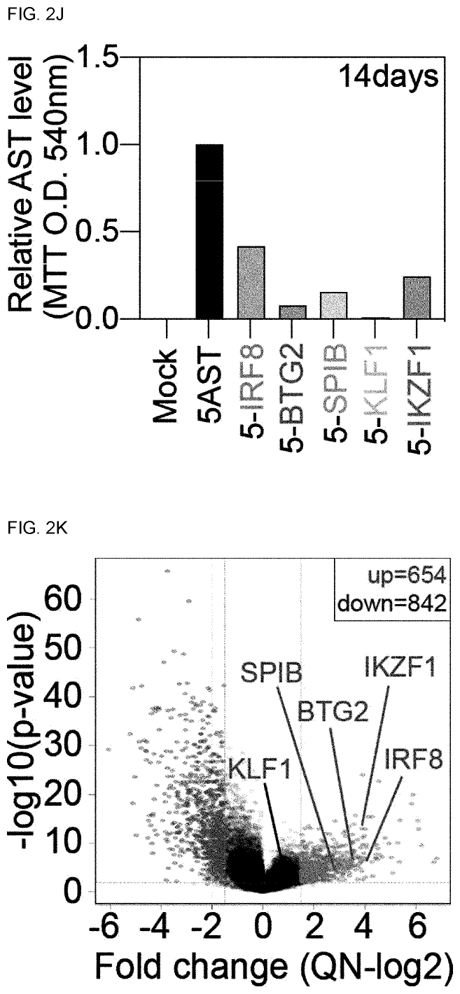

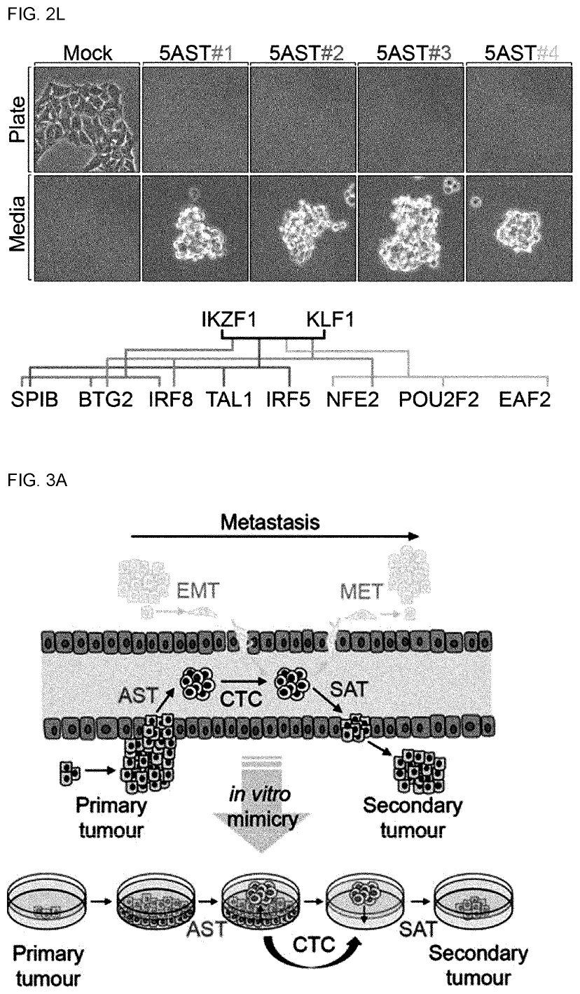

To evaluate the 20 AST candidate genes, HEK293A cells that stably express these genes were established using lentivirus. The transduced cells were seeded again and then screened with puromycin (4 mg/m L) 3 days later (FIG. 2a). Surprisingly, when the 20 AST candidate genes were introduced into adherent HEK293A cells, they were transformed to suspension cells [hereinafter, referred to as “induced suspension cells (iS cells)”] (FIGS. 2b and 2c). It was confirmed through LIVE/DEAD and competitive proliferation assays that puromycin-resistant iS-HEK293A cells have no defects in survival or proliferation (FIGS. 2d and 2e).

Next, the inventors of the present disclosure intended to investigate a minimal combination capable of inducing AST by testing general factors expressed in two independent iS-HEK293A cells. For this, 10 candidate factors (GATA1, IKZF1, IKZF3, SPIB, TAU, IRF8, EAF2, POU2F2, BTG2 and KLF1) that produce AST-induced cells when introduced into adherent HEK293A cells were identified (FIGS. 2f and 2g). Next, after removing each candidate gene from the 10 AST factors introduced to the adherent HEK293A cells, the degree of AST induction was measured. Among the 10 candidates, when any of IRF8, BTG2, SPIB, IKZF1 and KLF1 was removed, the degree of AST was decreased significantly. AST-reprogrammed iS cells could be produced with a combination of the 5 factors (FIGS. 2h-2i). When any of the 5 AST factors was removed, the transition of HEK293A cells to suspension cells was decreased significantly (FIG. 2g). This result suggests that the combination of the 5 AST factors plays a key role in reprogramming adhesion dependence. In addition, it was confirmed that a combination of the 5 AST factors including IKZF1 and KLF1 as essential factors is the key element (FIG. 2l).

In Vitro CTC Assay Mimicking Development of Circulating Tumor Cells (CTCs)

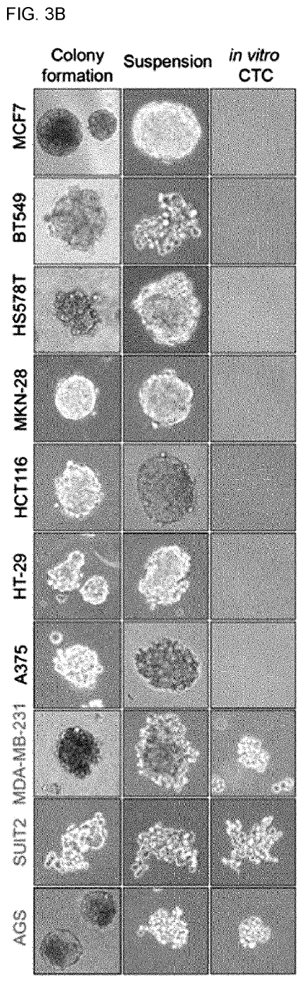

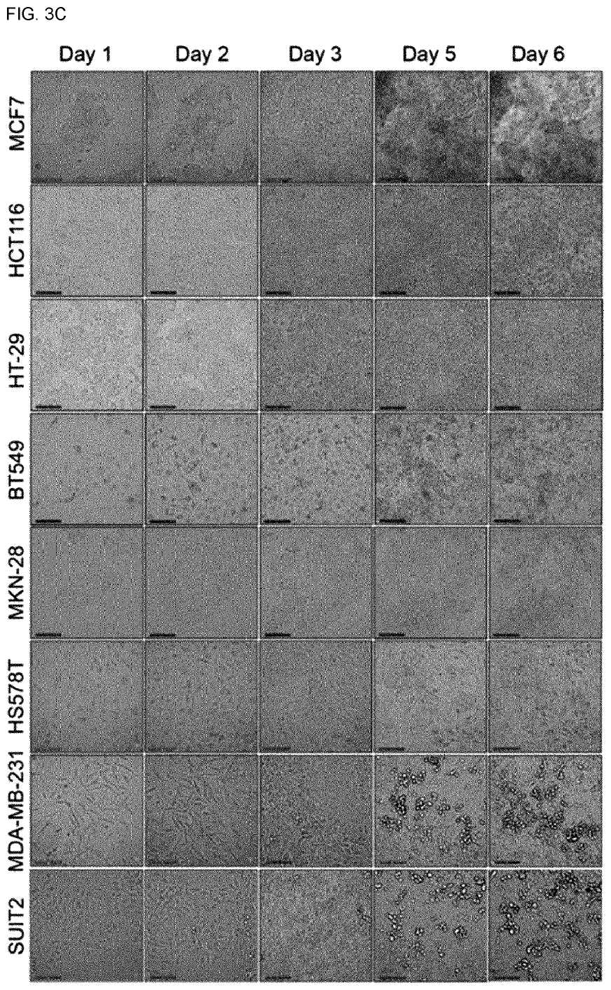

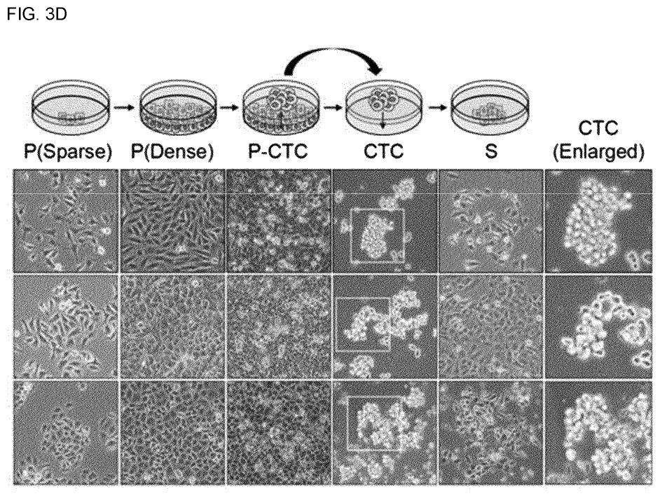

Until now, it is known that cancer metastasis is accompanied by EMT, or the morphological change of adherent cells. However, recent reports raise questions about the role of EMT in cancer metastasis. For metastasis to occur, cancer cells should circulate through the bloodstream as suspension cells. The mechanism of the development and function of these circulating tumor cells (CTCs), which is essential in metastasis, is not known well yet. The inventors of the present disclosure assumed that the AST factors screened in the present disclosure would play an important role in the development of CTCs and facilitation of cancer metastasis since they are widespread in tumor cells. In order to test this assumption, the inventors of the present disclosure developed a CTC formation assay as a new in-vitro assay platform, which can be applied as the most stringent parameter in measuring the malignancy and spread of cancer. The inventors of the present disclosure present a new concept of cancer metastasis which is entirely different from the existing EMT model. In this model, primary tumor cells are placed under stressful situations such as high cell density and AST is induced as a result. Then, the AST-induced tumors settle at the secondary sites through SAT induction (FIG. 3a). Surprisingly, although most cell lines were subjected to test assays such as colony formation assay and suspension assay, which are known to provide information about the aggressiveness and metastasis of cancer cells, only 3 out of the 14 tested cell lines survived and produced CTC-like cells (FIG. 3b). CTC formation assay in vitro was visualized by live-cell imaging. As shown in FIGS. 3c and 3d, when the cell density reached a very high density, some cells died completely and some survived but could not form CTC-like cells. Only 3 cells survived and produced CTC-like suspension cells.

Knockdown of AST Factors in Cancer Cell and Inhibition of CTC Production

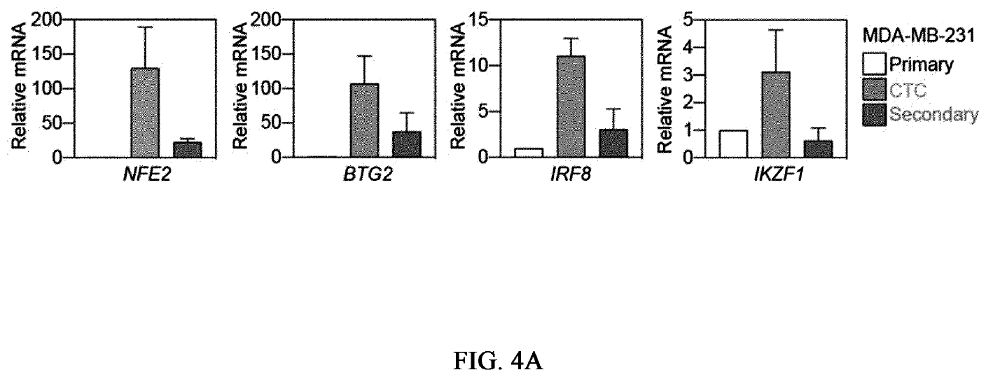

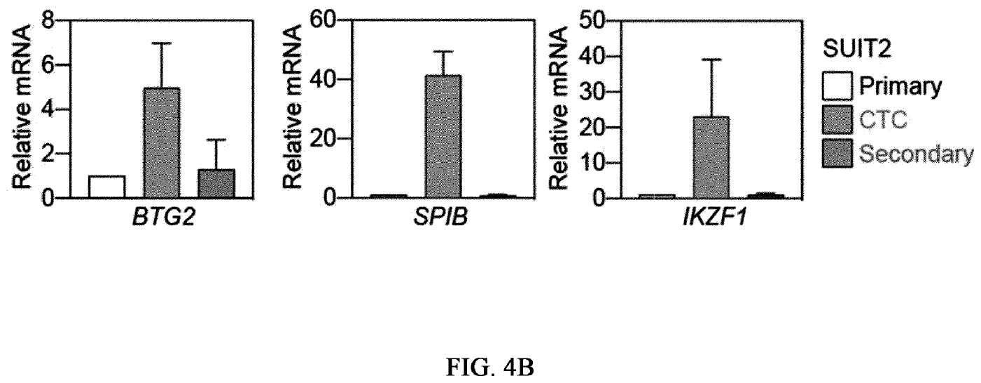

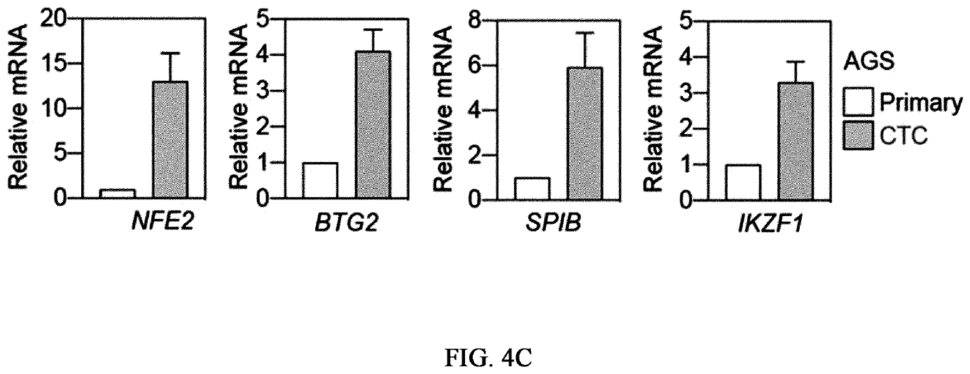

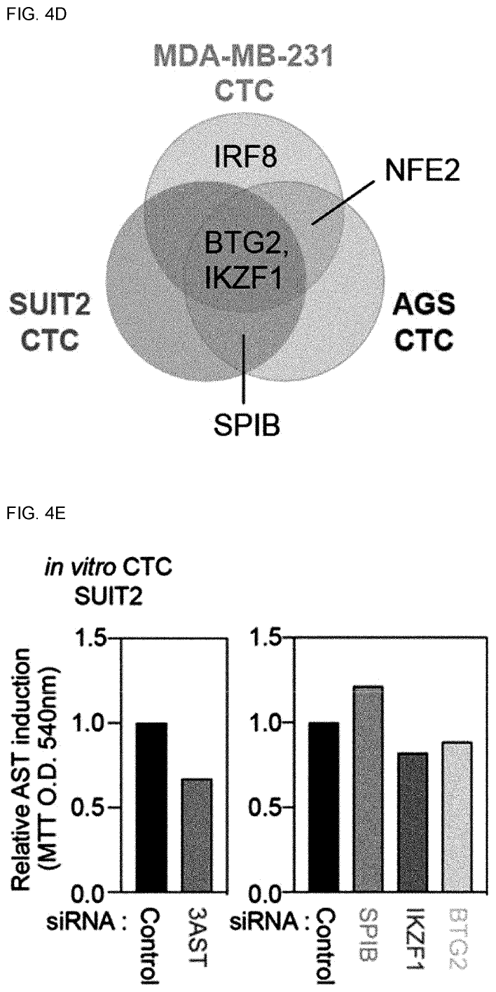

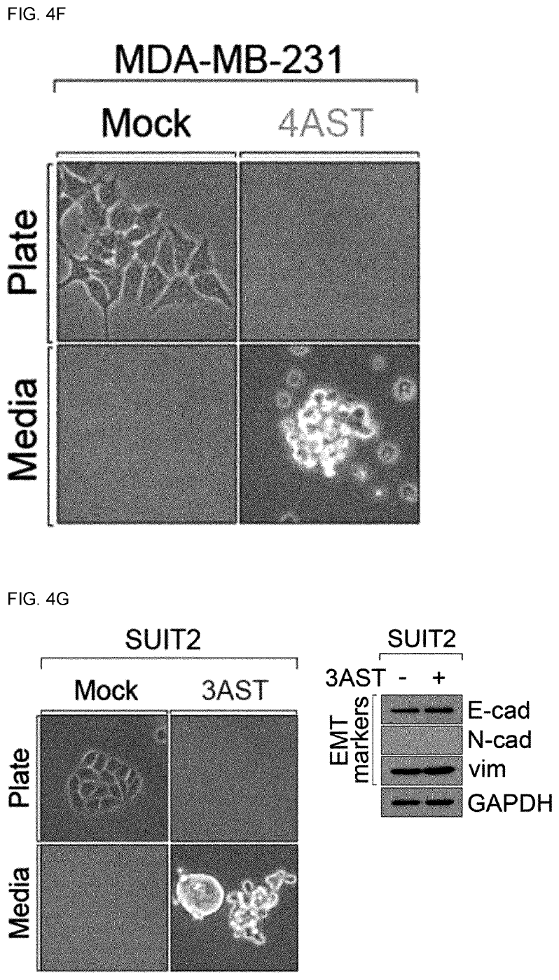

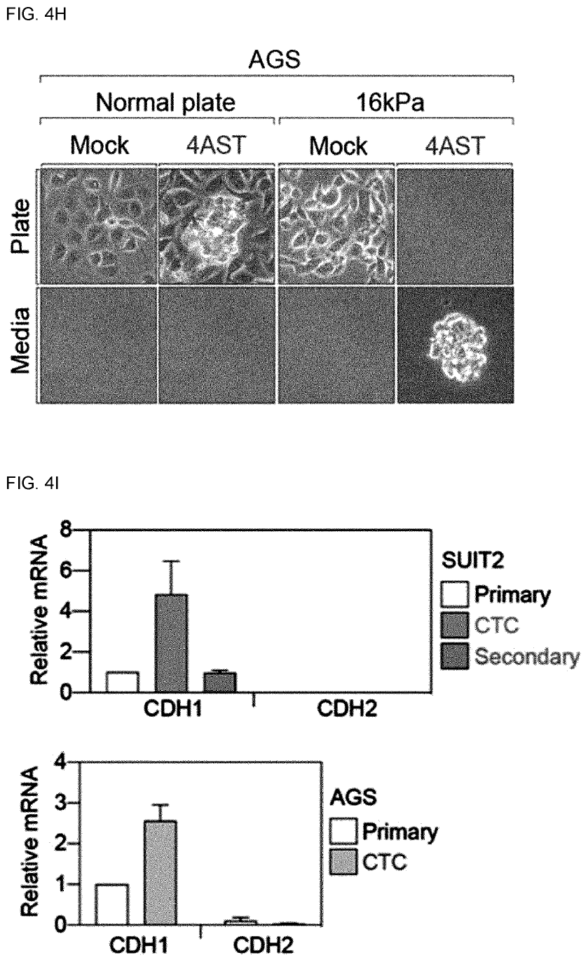

Next, the inventors of the present disclosure investigated the mRNA expression of several AST factors in the CTC-like cells of MDA-MB-231, SUIT2 and AGS cells. Surprisingly, the CTC-like cells of these cells showed abrupt expression of AST factors, i.e., NFE2, BTG2, IRF8 and IKZF1 in MDA-MB-231 cells, BTG2, SPIB and IKZF1 in SUIT2 cells, and NFE2, BTG2, SPIB and IKZF1 in in AGS cells (FIGS. 4a, 4b and 4c). It is noteworthy that different types of cancer cells show different combinations of AST factors, which suggests that various combinations of AST factors are involved in the regulation of adhesion dependence in different cell types. In conclusion, it can be seen that the AST factors not observed in primary tumors can be an effective target for inhibiting metastasis.

Next, the inventors of the present disclosure investigated whether the abrupt expression of AST factors in CTC-like cells induce such phenotype. For this, AST factors were knocked down in MDA-MB-231 and SUIT2 cells. As a result, it was confirmed that the functional loss of the AST factors resulted in diminished production of CTC-like cells (FIGS. 4d and 4e). In contrast, when the 5 AST factors expressed in 231-CTC and SUIT2 cells were introduced, AST-induced MCF10A and SUIT2 suspension cells could be obtained, respectively. This result indicates that the AST factors promote the production of CTC-like suspension cells (FIGS. 4f and 4g). Next, the inventors of the present disclosure investigated whether CTC formation assay is accompanied by EMT. Because SUIT2 and AGS cells are known as epithelial cells, the mRNA expression level of E-cadherin and N-cadherin in the CTC-like cells of SUIT2 and AGS cells was investigated. Surprisingly, the expression of E-cadherin in CTCs was increased contrary to expectation (FIG. 4h). In addition, as a result of investigating the expression of E-cadherin in SUIT2 suspension cells induced by artificial expression of AST factors, no change was observed, suggesting that EMT is not essential in AST (FIG. 4g).

TABLE 1
Sequence listings of AST factors and SAT factors

| SEQ ID NO | Gene |
|---|---|
| 1 | NFE2 |
| 2 | BTG2 |
| 3 | SPIB |
| 4 | IRF8 |
| 5 | RHOXF2 |
| 6 | IKZF3 |
| 7 | KLF2 |
| 8 | TAL1 |
| 9 | EAF2 |
| 10 | GFI1B |
| 11 | GATA1 |
| 12 | KLF1 |
| 13 | MYB |
| 14 | POU2F2 |
| 15 | AKNA |
| 16 | IKZF1 |
| 17 | SPI1 |
| 18 | IRF5 |
| 19 | TCF7 |
| 20 | SPI1 |
| 21 | TSC22D1 |
| 22 | VAX2 |
| 23 | SOX13 |
| 24 | ARNT2 |
| 25 | PPARG |
| 26 | BNC2 |
| 27 | HOXD8 |
| 29 | GLIS3 |
| 30 | FOXD8 |
| 31 | RARG |
| 32 | MEIS3 |
| 33 | TGFB1I1 |
| 34 | TBX3 |
| 35 | SOX9 |
| 36 | EPAS1 |
| 37 | TEAD2 |
| 38 | SNAI2 |
| 39 | TEAD1 |

Inhibition of Colonization Through Overexpression of AST Factors in Produced CTCs

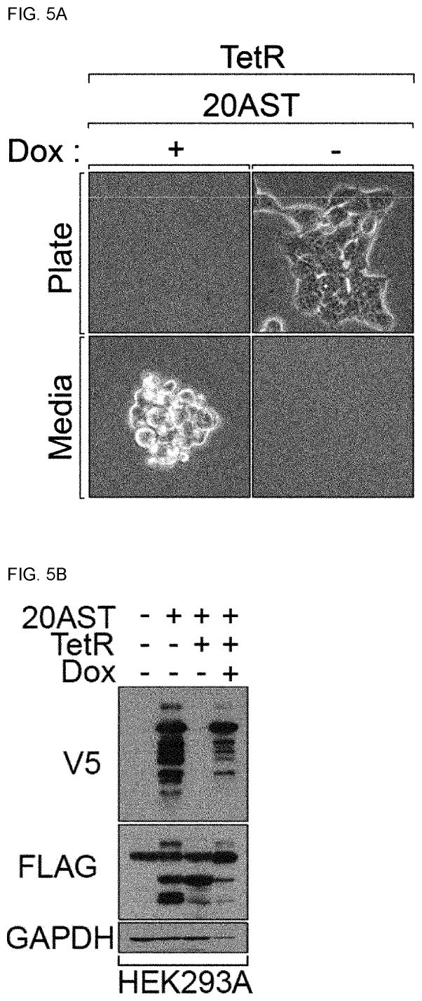

The inventors of the present disclosure investigated whether the CTCs produced through expression of the AST factors acquire adhesiveness again and are colonized when the AST factors are inhibited. For this, a plasm id expressing TetR, which is a Tet repressor protein, was introduced into HEK293 cells so that the expression of AST candidate factors was inhibited and they could be expressed only upon treatment with doxycycline. Interestingly, whereas TetR effectively inhibited the expression of several candidate factors and induction of AST, the treatment with doxycycline induced the expression of AST candidate genes and the development of iS-HEK293A cells. In addition, when the doxycycline was removed, the expression of the AST factors in the HEK293A cells was decreased and the cells were colonized as they acquired adhesiveness (FIGS. 5a and 5b).

While the specific exemplary embodiments of the present disclosure have been described in detail, it will be obvious to those having ordinary knowledge in the art that they are merely specific exemplary embodiments and the scope of the present disclosure is not limited by them. It is to be understood that the substantial scope of the present disclosure is defined by the appended claims and their equivalents.

We claim:

1.-20. (canceled)

21. A method for preventing or treating cancer in a subject in need thereof, comprising a step of administering a composition comprising,
an inhibitor of the expression of one or more gene selected from a group consisting of IKZF1, KLF1, IRF8, BTG2, SPIB, GATA1, IKZF3, TALL EAF2, POU2F2, KLF2, SP11, NFE2, AKNA, IRF5, TCF7, RHOXF2, MYB, BCL11A and GFI1B; or
a nucleotide of one or more gene selected from a group consisting of IKZF1, KLF1, IRF8, BTG2, SPIB, GATA1, IKZF3, TALL EAF2, POU2F2, KLF2, SP11, NFE2, AKNA, IRF5, TCF7, RHOXF2, MYB, BCL11A and GFI1B; as an active ingredient.

22. The method according to claim 21, wherein the composition comprises expression inhibitors of the BTG2 and IKZF1 genes.

23. The method according to claim 22, wherein the composition further comprises expression inhibitors of the NFE2, IRF8 and SPIB genes.

24. The method according to claim 23, wherein the composition further comprises expression inhibitors of the GATA1, IKZF3, TALL EAF2 and POU2F2 genes.

25. The method according to claim 21, wherein the cancer is cancer before production of circulating tumor cells (CTCs), and the composition inhibits the production of circulating tumor cells.

26. The method according to claim 21, the cancer wherein circulating tumor cells (CTCs) have been produced.

27. The method according to claim 26, wherein the composition inhibits the colonization of the produced circulating tumor cells.

28. A composition for diagnosing the metastasis or recurrence of cancer, comprising an agent for measuring the expression of one or more gene selected from a group consisting of IKZF1, KLF1, IRF8, BTG2, SPIB, GATA1, IKZF3, TALL EAF2, POU2F2, KLF2, SP11, NFE2, AKNA, IRF5, TCF7, RHOXF2, MYB, BCL11A, GFI1B, TSC22D1, VAX2, SOX13, ARNT2, PPARG, BNC2, HOXD8, GLIS3, FOXD8, RARG, MEIS3, TGFB111, TBX3, SOX9, EPAS1, TEAD2, SNA12 and TEAD1 as an active ingredient.

29. The composition according to claim 28, wherein, if the expression of one or more gene selected from a group consisting of IKZF1, KLF1, IRF8, BTG2, SPIB, GATA1, IKZF3, TALL EAF2, POU2F2, KLF2, SP11, NFE2, AKNA, IRF5, TCF7, RHOXF2, MYB, BCL11A and GFI1B has increased, it is determined that the risk of cancer metastasis or recurrence has increased due to production of circulating tumor cells (CTCs).

30. The composition according to claim 28, wherein, if the expression of one or more gene selected from a group consisting of TSC22D1, VAX2, SOX13, ARNT2, PPARG, BNC2, HOXD8, GLIS3, FOXD8, RARG, MEIS3, TGFB111, TBX3, SOX9, EPAS1, TEAD2, SNA12 and TEAD1 has decreased, it is determined that the risk of cancer metastasis or recurrence has increased due to production of circulating tumor cells (CTCs).

31. The composition according to claim 28, wherein the composition further comprises agents for measuring the expression of the BTG2 and IKZF1 genes.

32. The composition according to claim 31, wherein the composition further comprises agents for measuring the expression of the NFE2, IRF8 and SPIB genes.

33. The composition according to claim 32, wherein the composition further comprises agents for measuring the expression of the GATA1, IKZF3, TALI, EAF2 and POU2F2 genes.

34. A method for screening an inhibitor of the production of circulating tumor cells (CTCs) or an inhibitor of the colonization of produced circulating tumor cells, comprising:
(a) a step of contacting a test substance with a biological sample comprising one or more gene selected from a group consisting of IKZF1, KLF1, IRF8, BTG2, SPIB, GATA1, IKZF3, TALI, EAF2, POU2F2, KLF2, SP11, NFE2, AKNA, IRF5, TCF7, RHOXF2, MYB, BCL11A, GFI1B, TSC22D1, VAX2, SOX13, ARNT2, PPARG, BNC2, HOXD8, GLIS3, FOXD8, RARG, MEIS3, TGFB111, TBX3, SOX9, EPAS1, TEAD2, SNA12 and TEAD1 or a protein encoded thereby; and
(b) a step of measuring the expression or activation of the gene or the protein in the sample, wherein
if the expression or activation of one or more gene selected from a group consisting of IKZF1, KLF1, IRF8, BTG2, SPIB, GATA1, IKZF3, TALL EAF2, POU2F2, KLF2, SP11, NFE2, AKNA, IRF5, TCF7, RHOXF2, MYB, BCL11A and GFI1B or a protein encoded thereby has decreased or if the expression or activation of one or more gene selected from a group consisting of TSC22D1, VAX2, SOX13, ARNT2, PPARG, BNC2, HOXD8, GLIS3, FOXD8, RARG, MEIS3, TGFB111, TBX3, SOX9, EPAS1, TEAD2, SNA12 and TEAD1 or a protein encoded thereby has increased, the test substance is determined as an inhibitor of the production of circulating tumor cells (CTCs), and
if the expression or activation of one or more gene selected from a group consisting of IKZF1, KLF1, IRF8, BTG2, SPIB, GATA1, IKZF3, TALL EAF2, POU2F2, KLF2, SP11, NFE2, AKNA, IRF5, TCF7, RHOXF2, MYB, BCL11A and GFI1B or a protein encoded thereby has increased or if the expression or activation of one or more gene selected from a group consisting of TSC22D1, VAX2, SOX13, ARNT2, PPARG, BNC2, HOXD8, GLIS3, FOXD8, RARG, MEIS3, TGFB111, TBX3, SOX9, EPAS1, TEAD2, SNA12 and TEAD1 or a protein encoded thereby has decreased, the test substance is determined as an inhibitor of the colonization of produced circulating tumor cells.

35. The method according to claim 34, wherein the biological sample comprises cancer cells.
